(19) United States
(12) Patent Application Publication
(10) Pub. No.: US 2022/0193310 A1
(43) Pub. Date: Jun. 23, 2022

(54) DUAL AGENT NANOPARTICLE COMPOSITION FOR COATING MEDICAL DEVICES

(71) Applicant: THE CLEVELAND CLINIC FOUNDATION, Cleveland, OH (US)

(72) Inventor: Vinod D. Labhasetwar, Solon, OH (US)

(21) Appl. No.: 17/556,093

(22) Filed: Dec. 20, 2021

Related U.S. Application Data
(60) Provisional application No. 63/127,697, filed on Dec. 18, 2020.

(51) Int. Cl. A61L 29/16; A61L 31/16; A61L 31/12; A61L 29/12; A61K 31/436; A61K 31/337

(52) U.S. Cl. CPC A61L 29/16; A61L 31/16; A61L 31/129; A61L 29/126; A61K 31/436; A61K 31/337; A61L 2420/08; A61L 2300/802; A61L 2300/416; A61L 2300/216; A61L 2420/02; A61L 2420/04; A61L 2400/12

(57) ABSTRACT

A pharmaceutical composition is described. The pharmaceutical composition includes a polymeric coating composition comprising polymeric nanoparticles dispersed within a polymeric matrix, wherein the polymeric nanoparticles include a first therapeutic agent and a second therapeutic agent. Implantable medical devices coated with the pharmaceutical composition, methods of coating an implantable medical device with the pharmaceutical composition, and methods of treating vascular disease using the pharmaceutical composition are also described.

### CROSS-REFERENCE TO RELATED APPLICATIONS

This application claims priority to U.S. Provisional Application Ser. No. 63/127,697, filed on Dec. 18, 2020, which is hereby incorporated by reference in its entirety.

### BACKGROUND

The number of patients with severe lower extremity peripheral artery disease (PAD) and potentially limb threatening critical limb ischemia is on the rise worldwide as a result of aging population and a growing number of patients with vascular disease due to diabetes and other underlined inflammatory conditions and metabolic disorders. According to recent estimates, PAD afflicts some 27 million people in the US and Europe. About 20% have disease severe enough to warrant intervention. The femoropopliteal arteries, which run from mid-thigh to below the knee and comprise the superficial arteries and the smaller popliteal arteries are the most common blood vessels afflicted with PAD, accounting for about 60% of all lower-extremity occlusions. In peripheral vasculature, acute failure after balloon angioplasty has been reported in 30 to 40% cases, and stent failure rate is 20 to 35% range. Thus, there is an urgent clinical unmet need and growing demand for effective treatment options for PADs.

Restenosis pertains to blood vessel that has received treatment to clear the blockage but subsequently becomes re-narrowed, leading to restricted blood flow. Restenosis is a common adverse event of endovascular procedures such as stenting or balloon angioplasty that are used to open-up clogged blood vessels primarily because of atherosclerosis. When a stent is used and restenosis occurs, this is called in-stent restenosis (ISR). If it occurs following balloon angioplasty, this is called post-angioplasty restenosis (PARS). Restenosis could occur in an artery, or other blood vessels, or possibly a vessel within an organ that have undergone the interventional procedures.

Vascular intervention such as stenting or balloon angioplasty used to widen or unblock a blood vessel usually has a long-lasting beneficial effect for the patient. However, in some cases, the procedure itself can cause re-narrowing of the vessel, or restenosis. Angioplasty, also called percutaneous transluminal angioplasty (PTA), is commonly used to treat blockages of the coronary (PTCA), carotid, hepatic, renal, or peripheral arteries (such as in the limbs). The balloon inserted into the narrowing ‘smashes’ the cholesterol plaques (atherosclerosis) against the artery walls, thus widening the size of the lumen and increasing blood flow. However, the action damages the artery walls, and they respond by using physiological mechanisms to repair the damage. In this process, smooth muscle cells (SMC) which are a part of a blood vessel (forms the middle layer in a blood vessel, called the media), are stimulated due to exposure to growth factors present in the blood and inflammatory cells that migrate to the injured site. These events cause SMC to proliferate into the lumen of the blood vessel, a process of restenosis, thus re-blocking the blood flow to the target tissue/organ. In normal or healthy blood vessel, endothelium or innermost lining of the blood vessel acts as a barrier and protects SMC but this protective endothelial lining is disrupted in diseased blood vessel or following an intervention.

A stent is a mesh, tube-like structure deployed with an angioplasty balloon as a delivery mechanism. The stent not only support a weakened blood vessel called aneurysm, but also is used as a local drug delivery mechanism. The drug is delivered up to 180 days and stent is permanently left behind and not contributing to either drug delivery or vessel structural support. However, the vessel can react to the stent, perceive it as a foreign body, and respond by mounting an inflammatory immune response which leads to further narrowing near or inside the stent.

Damage to the blood vessel wall either due to angioplasty or stenting triggers physiological response that can be divided into two stages. The first stage that occurs immediately after tissue trauma is thrombosis. A blood clot forms at the site of damage and further hinders blood flow. The patients following vascular intervention are treated with anticoagulants/antiplatelet agents to prevent thrombosis. In certain cases, vessel overstretch during mechanical opening of the vessel due to intervention results in recoiling of the blood vessel or collapse. These events are accompanied by an inflammatory immune response that triggers the second stage, tends to occur days to months after intervention, and is the result of proliferation of cells in the media, where SMC are located in the vessel wall. This proliferative cell mass formed in the lumen of a blood vessel is known as Neointimal Hyperplasia (NIHA). Rates of restenosis differ between devices (e.g., stent-grafts, balloon angioplasty, etc.) and location of procedure (e.g., centrally located in the heart, such as the coronary artery, or in peripheral vessels such as the popliteal artery in the leg, the pudendal artery in the pelvis, or the carotid artery in the neck). The response also depends on patient's overall health and age.

To prevent or minimize proliferation of SMC, stents are coated with antiproliferative drugs—these stents are known as drug eluting stents (DES). The data indicate that DES are better than bare metal stents (BMS) in preventing or delaying restenosis. Thus, DES provide physical support to weakened blood vessel in addition to delivering antiproliferative drug. However, the metal left behind after the coated drug is eluted can trigger inflammatory response that can cause proliferation of SMC and restenosis. If restenosis occurs within a stent, it is known as in-stent stenosis. Restenosis also occurs at either the proximal or distal end of the stent. Thus, the patients receiving DES are at a high risk of late thrombosis and restenosis.

Bioresorbable scaffold, acting like a stent where the polymer used for the fabrication of stent is resorbed by the tissue to address the issue with metal stents has been tested. However, such stents failed in long-term human studies and caused more mortality than metal-based DES. The reason attributed to failure of bioresorbable stent is the lack of full stent deployment against the vessel and poor apposition to the vessel.

Because of the above reasons, over the past 5 years, vascular blockage is being increasingly treated with drug-coated balloons (DCBs), which is a balloon coated with antiproliferative drugs to prevent restenosis. DCB are inflated to achieve transfer of the coated drugs into the vessel wall. DCB treatment, unlike stents, does not leave an implant in the body and repeat procedure is feasible if necessary, such as when there is incomplete opening of the clogged blood vessel during the first attempt or restenosis. In addition, DCB can be navigated through small and complex or long blood vessels which with stents is not feasible due to large strut-to-vessel ratio.

The DCB available in the US market are: Medtronic's In.PACT Admiral, C. R. Bard's Lutonix, Boston Scientific's Ranger, Philips' Stellarex. These DCBs are available only for a superficial femoral artery indication, and none are approved for below the knee/critical limb ischemia or coronary indications. The key differentiating aspects of DCBs are what drug is used and how it is attached to the balloon surface. All the current DCBs use an excipient to hold the drug in place, so it does not immediately wash off in the blood. Philips' Stellarex DCBs feature EnduraCoat technology, a coating consisting of a polyethylene glycol excipient with amorphous and crystalline paclitaxel particles dispersed in it. In.PACT balloon uses urea as a substrate for coating of paclitaxel. Lutonix device is also a paclitaxel-coated balloon; in this the main excipients contain polysorbate (surfactant) and sorbitol. Ranger from Boston Scientific uses photopolymerizable polymer in which paclitaxel is embedded.

These DCB contain crystalline or a combination of crystalline and amorphous paclitaxel. The type of crystal form (i.e., amorphous, anhydrous, crystalline) have different dissolution profiles, and plays a role in the pharmacokinetics of drug uptake and retention. Following balloon inflation, crystalline form of paclitaxel penetrates the vessel wall because of sharp, needle-like edges whereas amorphous form diffuses into the wall. Crystalline form of paclitaxel dissolves slowly, thus prolonging the drug retention whereas amorphous form dissolves relatively rapidly that provides initial dose of the drug. The FDA cleared the Medtronic In.Pact AV DCB for the treatment of failing AV access in patients with end-stage renal disease (ESRD) undergoing dialysis. Over time, vessel restenosis limits the ability to use AV fistulae effectively.

MedAlliance's Selution SLR 0.014 DCB is sirolimus coated balloon that uses MicroReservoirs made from biodegradable polymer, which provide controlled and sustained drug release. A bioadhesive film holds MicroReservoirs against the wall of the blood vessel, and the MicroReservoirs do not penetrate the vessel-wall; the drug encapsulated in MicroReservoirs is released and diffuses into the vessel wall through a passive diffusion. Concept Medical Inc. MagicTouch™ sirolimus DCB is nanocrystal-based formulation encapsulated within a phospholipid nano-carrier. The nano-crystals are transported into the tissue using lipid nano-carrier. The nano-carrier releases nanocrystals upon localization in the tissue. Orchestra BioMed, in a distribution partnership with Terumo, developed Virtue Sirolimus-Eluting Balloon (SEB). The SEB is a double balloon technology and sirolimus encapsulated/solubilized in micellar formulation is delivered to the target vessel wall through the micropores in outer balloon wall.

There is growing concern with DCB coated with crystalline paclitaxel as large-sized crystals (several microns to millimeter) that flow downstream causing embolism and tissue necrosis. This concern has promoted the FDA to issue a warning letter. Thus, there remains a need for improved compositions and methods for delivering drugs from implantable medical devices, and in particular drug coated balloons.

### SUMMARY

The present invention describes a composition and formulation of nanoparticles containing one or more antiproliferative drugs. The composition also contains agents that can promote vascular repair and re-endothelization. In some embodiments, surface functionalization of nanoparticles with one or more modifying agents can be used to facilitate cellular/tissue uptake of nanoparticles and the encapsulated therapeutics. In addition to the encapsulated therapeutics, nanoparticles can contain imaging agent, plasticizer and/or fatty acid to impart different features (e.g., to modulate release or imaging capability) to nanoparticle formulations. A composition and method of coating onto medical devices with controlled elution profile of the coated nanoparticles is also described. In addition to nanoparticles, the coating composition can include solubilized drug(s), imaging agent(s), plasticizer, lubricant, and/or sugar. Medical devices such as balloons, stents, catheters, surgical mesh, sutures, bandages, vascular grafts, implants, catheter leads, fistula, perfusion catheters, etc. can be coated using the pharmaceutical composition and methods disclosed herein.

Another aspect of the invention provides a method to coat an implantable medical device (e.g., a balloon or stent) to deliver a therapeutic agent encapsulated in functionalized nanoparticles to the diseased artery following vascular intervention (e.g., angioplasty) to prevent restenosis. Functionalized nanoparticles result in efficient delivery and sustained retention of therapeutics in cells and tissue. For example, paclitaxel can be encapsulated into functionalized nanoparticles, but other therapeutics (hydrophobic/hydrophilic and biological agents) as well can be delivered either alone or in combination. In addition, nanoparticles with different size/surface charge, release profile, composition, drug load, etc. can be formulated and used alone or in combination. Drug-loaded nanoparticles are dispersed in an aqueous polymer (e.g., polyvinyl alcohol (PVA)) solution, also referred to as the coating composition, is coated onto a balloon layer-by-layer using dip-coating method; with last few coatings with PVA solution without nanoparticles to minimize wash out of nanoparticle layer(s) during transit and to maximize the availability of the coated nanoparticles to the target vessel. In an alternative approach, a film of PVA with nanoparticles is casted separately using solvent evaporation method and then wrapped around a balloon in single or multiple layers. The wrapped film with drug-loaded nanoparticles could be further wrapped with PVA film without nanoparticles or coated with PVA solution to minimize wash out effect. The number of PVA coats or concentration of PVA used can be varied to control the lag phase for the release of the nanoparticle layer. This feature is critical for control as the transit time for DCB could vary depending upon the entry point for DCB and the target vessel site.

In addition to nanoparticles and PVA, the coating composition contains a plasticizer, a lubricant, and a sugar. Amount, ratios or a combination of these agents can be varied and optimized in the coating composition per clinical unmet need and indication.

The coating procedure and functionalized nanoparticles loaded with dual active pharmaceutical agents offers several advantages over the In.PACT, Stellarex or Lutonix DCBs. Uniform coating of nanoparticles that can result in consistent and efficient delivery of therapy to the target blood vessel. Nanoparticles are ˜300 nm in diameter (hydrodynamic means with associated water; actual diameter by TEM is ˜200 nm), (FIG. 1) significantly smaller than the drug crystals in the eluted samples seen from the In.PACT catheter. Functionalized nanoparticles (modified with poly-1-lysine) helps in enhancing cellular/arterial uptake and retention of therapeutics (based on previous data; see U.S. Pat. No. 8,865,216. This is expected to sustain drug levels in the arterial wall which otherwise with the currently used DCB shows rapid exponential decay with time. Downstream flow of paclitaxel will be significantly reduced, hence non-target tissue toxicity. Since the drug is encapsulated and released slowly, there won't be drug dumping effect seen with drugs alone. Further, drug crystals are quite large to cause arterial occlusion downstream which is not an issue with nanoparticles due to their small size that can easily pass through fine blood capillaries in the tissue.

### BRIEF DESCRIPTION OF THE FIGURES

FIGS. 1A-1C provide graphs showing a particle size analysis of A) Paclitaxel-B) Sirolimus- and C) Combination of paclitaxel and sirolimus-loaded nanoparticles. Nanoparticles were dispersed in water and particle size was measured using dynamic light scattering (DLS) method. Mean hydrodynamic diameter of paclitaxel-loaded nanoparticles=248 nanometer (nm) with polydispersity index (PI)=0.056; sirolimus-loaded nanoparticle, mean diameter=255.4 nm with PI=0.100; and mean diameter of paclitaxel+sirolimus loaded nanoparticles=221.8 nm with PI=0.063. All the formulations of nanoparticles have positive zeta potential because of functionalization with PLL+PVA or PLL+PVA+cationic surfactant. Nanoparticles formulated without functionalization carry negative zeta potential.

FIGS. 2A and 2B provides graphs showing the differential scanning calorimetric analysis of A) pure paclitaxel and B) following encapsulation in nanoparticles. The absence of melting peak at 240° C. seen in pure paclitaxel was absent following encapsulation in nanoparticles indicating amorphous nature of the encapsulated paclitaxel.

FIGS. 3A-3D provide images of nanoparticle coated balloon and stent. For optimal imaging, a near infrared dye along with nanoparticles were encapsulated. The dye signal in nanoparticles can be captured using Maestro Optical Imaging. A-a): Balloon coated with nanoparticles as seen using Maestro; B-b) Stent coated with nanoparticles containing near-infrared dye. There is distinct difference in the optical signal of the coated segment of the stent vs. uncoated segment of the stent as seen following optimal imaging with Maestro. B-c) Top view of the nanoparticle coated stent indicating coating of inside and outside of the stent. C-d) Scanning electron micrographic picture of the nanoparticle coated balloon. There is distinct difference in the uncoated and coated balloon. Black arrows indicate the presence of nanoparticles in the coating.

FIG. 4 provides graphs and images showing quantification of signal of the coating. Nanoparticles encapsulating active pharmaceutical agent and near-infrared dye in the same formulation following coating on to a balloon. The segmental analysis of the coated balloon for optical signal intensity can be carried out to determine consistency in the coating. The data show uniform coating along the length of the balloon. There is no background signal from the uncoated balloon, indicating that the signal captured is due to the nanoparticle coating onto the balloon.

FIG. 5 provides graphs showing the elution of balloon coated nanoparticles. Balloon coated nanoparticles encapsulating active pharmaceutical ingredient (API) and near-infrared dye were eluted in water. The topcoat delays the elution by ˜30 seconds and complete elution was achieved in about 3 minutes. Insignificant amount of the coated nanoparticles was left onto the balloon after elution.

FIG. 6 provides an image showing a transmission electron micrograph of the nanoparticle coated balloon shows basecoat, nanoparticle coat and topcoat. Base- and topcoats do not contain nanoparticles but PVA (without nanoparticles). The topcoat prevents washout effect during transit and protects the underlined nanoparticle coat containing API.

FIG. 7 provides a graph and flowchart of elution of coating in physiologic buffer to mimic in vivo conditions. The added sugar in the coating (reconstitution buffer) can control the elution of the coated nanoparticles. This is critical as transit time could vary, depending upon the intended target vessel and entry point of the balloon. By controlling the topcoat, one can minimize the wash out effect of the nanoparticles.

FIG. 8 provides images showing nanoparticles encapsulating sirolimus (Nano-Rap) can be coated onto balloon in the same way as paclitaxel coated balloons.

FIG. 9 provides graphs and images showing the results when peracetic acid was used for sterilization. There is no particle aggregation, change in particle size or zeta potential or the extracted drug from nanoparticles as it did not alter the retention time.

FIG. 10 provides a graph showing the results when paclitaxel coated balloons were deployed in pig blood vessels and the arteries were harvested for drug level analysis at different time points. Following extraction, the drug levels in the tissue were determined by LCMS. The data show sustained drug tissue retention.

FIGS. 11A-11D provide graphs showing that to determine the efficacy of the treatment in primary human vascular smooth muscle cells with paclitaxel alone, sirolimus alone and in combination, both is solution for the initial evaluation and following encapsulation of both the API either in separate nanoparticle formulations or co-encapsulated into a single nanoparticle formulation. Cell viability was determined using CyQUANT™ NF Cell Proliferation Assay kit (Cat #C35006, ThermoFisher Scientific). Fluorescent readings were taken by a microplate reader (Cytation 5, BioTek Instruments Inc., Vermont). Percent cell inhibition was calculated with respect to the cells that did not receive any treatment. A) Dose-response effect following treatment of cells with Paclitaxel (PTX) or Sirolimus (SRL) in solution. From the dose-response study, IC50 for each drug was determined. IC₅₀ is the dose that is required to achieve 50% inhibition of cell proliferation with respect to untreated cell. The data show significantly higher dose of SRL is required than PTX to achieve IC50 (29066 vs. 1156 ng/ml). B) Addition of 10% PTX in the combination treatment significantly reduces the IC₅₀. This effect is highly synergistic as evident from the combination index. C and D) Further study was carried out with nanoparticles encapsulating both the drugs (PTX+SRL) in the same nanoparticle formulation and the results were compared with PTX-alone or SRL alone encapsulated nanoparticles. The data show that nanoparticles encapsulating both the drugs are effective in achieving inhibition of cell proliferation at significantly lower dose as compared to the individual drug encapsulated nanoparticles.

FIGS. 12A and 12B show graphs and images of the flow cytometric analysis of the cells treated either with PTX or SRL and combination was carried out to determine the dominant mechanism of action of the combination treatment on inhibition of cell proliferation. A) The combination treatment shows the same pattern of cell-cycle arrest (G0/G1) as sirolimus/rapamycin alone treatment whereas PTX alone treatment shows more cells in G2/M which then progresses to apoptosis and death. In the above study, the combination dose was ˜14-fold lower than SRL alone dose, suggesting that a small dose of PTX in the combination (PTX:SRL ratio 1:9) treatment boosts the effect of SRL. B) The photograph of the cells treated with the combination of PTX: SRL, (1:9 w/w), despite significant efficacy in inhibiting cell proliferation, retains the cellular structure (actin). This confirms that at this ratio of drug combination, the effect is due to cell-cycle arrest (cytostatic).

### DETAILED DESCRIPTION

The present invention provides a pharmaceutical composition that includes a polymeric coating composition comprising polymeric nanoparticles dispersed within a polymeric matrix, wherein the polymeric nanoparticles include a first therapeutic agent and a second therapeutic agent. Implantable medical devices coated with the pharmaceutical composition, methods of coating an implantable medical device with the pharmaceutical composition, and methods of treating vascular disease using the pharmaceutical composition are also provided.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. All publications, patent applications, patents, and other references mentioned herein are incorporated by reference in their entirety.

### Definitions

As used in the description of the invention and the appended claims, the singular forms “a,” “an,” and “the” are intended to include the plural forms as well, unless the context clearly indicates otherwise. In addition, the recitations of numerical ranges by endpoints include all numbers subsumed within that range (e.g., 1 to 5 includes 1, 1.5, 2, 2.75, 3, 3.80, 4, 5, etc.).

As used herein, the terms “alkyl”, “alkenyl”, and the prefix “alk-” are inclusive of straight chain groups and branched chain groups and cyclic groups, e.g., cycloalkyl and cycloalkenyl. Unless otherwise specified, these groups contain from 1 to 20 carbon atoms, with alkenyl groups containing from 2 to 20 carbon atoms. In some embodiments, these groups have a total of at most 10 carbon atoms, at most 8 carbon atoms, at most 6 carbon atoms, or at most 4 carbon atoms. Lower alkyl groups are those including at most 6 carbon atoms. Examples of alkyl groups include haloalkyl groups and hydroxyalkyl groups. The number of carbon atoms can be indicated using the letter “C” followed by the number of carbon atoms present. For example, C12 refers to 12 carbon atoms.

“Treating”, as used herein, means ameliorating the effects of, or delaying, halting, or reversing the progress of a disease or disorder. The word encompasses reducing the severity of a symptom of a disease or disorder and/or the frequency of a symptom of a disease or disorder.

The language “effective amount” or “therapeutically effective amount” refers to a nontoxic but sufficient amount of the composition used in the practice of the invention that is effective to stimulate endothelial cell growth at the site of nanoparticle delivery. The desired treatment may be prophylactic and/or therapeutic. That result can be reduction and/or alleviation of the signs, symptoms, or causes of a disease or disorder, or any other desired alteration of a biological system. An appropriate therapeutic amount in any individual case may be determined by one of ordinary skill in the art using routine experimentation.

A “subject”, as used therein, can be a human or non-human animal. Non-human animals include, for example, livestock and pets, such as ovine, bovine, porcine, canine, feline and murine mammals, as well as reptiles, birds and fish. Preferably, the subject is human.

The term “biodegradable” as used herein refers to a polymer that can be broken down by either chemical or physical process, upon interaction with the physiological environment subsequent to administration, and erodes or dissolves within a period of time, typically within days, weeks or months. A biodegradable material serves a temporary function in the body, and is then degraded or broken into components that are metabolizable or excretable.

“Biocompatible,” as used herein, refers to any material that does not cause injury or death to the animal or induce an adverse reaction in an animal when placed in intimate contact with the animal's tissues. Adverse reactions include for example inflammation, infection, fibrotic tissue formation, cell death, or thrombosis. The terms “biocompatible” and “biocompatibility” when used herein are art-recognized and mean that the referent is neither itself toxic to a host (e.g., an animal or human), nor degrades (if it degrades) at a rate that produces byproducts (e.g., monomeric or oligomeric subunits or other byproducts) at toxic concentrations, does not cause prolonged inflammation or irritation, or does not induce more than a basal immune reaction in the host. It is not necessary that any subject composition have a purity of 100% to be deemed biocompatible. Hence, a subject composition may comprise 99%, 98%, 97%, 96%, 95%, 90% 85%, 80%, 75% or even less of biocompatible agents, e.g., including polymers and other materials and excipients described herein, and still be biocompatible.

### Pharmaceutical Nanoparticle Compositions

In one aspect, the present invention provides a pharmaceutical composition. The pharmaceutical composition includes a polymeric coating composition comprising polymeric nanoparticles dispersed within a polymeric matrix, wherein the polymeric nanoparticles include a first therapeutic agent (e.g., paclitaxel) and a second therapeutic agent (e.g., sirolimus). Dispersing the polymeric nanoparticles within a polymeric matrix provides several advantages, such as providing control over time and duration of release of the polymeric nanoparticles from the coating. Including the therapeutic agents in nanoparticles also allows for drugs that are relatively non-soluble to be dispersed and retained in a coating including the pharmaceutical composition. The term “dispersed,” as used herein, can refer to both even, gradient, and uneven dispersion of the nanoparticles within the polymeric matrix. Note that while the polymer matrix including the nanoparticles is referred to as a “polymer coating composition,” that it can also be used for applications outside of coating an implantable medical device.

### Nanoparticles

The pharmaceutical compositions include nanoparticles for efficient and timed-release delivery of therapeutics. A variety of release kinetics are contemplated for the timed release of pharmaceutical agents from the nanoparticle, including bi- or multi-phase release, such as an initial fast release followed by a slower subsequent release phase or delay the initial release for a certain period of time and then rapid or sustained release. For example, the release may include dissociation of the pharmaceutical agent from the nanoparticle rapidly within seconds or minutes followed by further sustained release over a period of at least 2, 4, 6, 8 or more hours to weeks and months. Such longer-term release can be referred to as sustained or prolonged release. Such release kinetics may be advantageous in certain circumstances, e.g., where sustained action is desired, in comparison with, e.g., an injection of free enzyme.

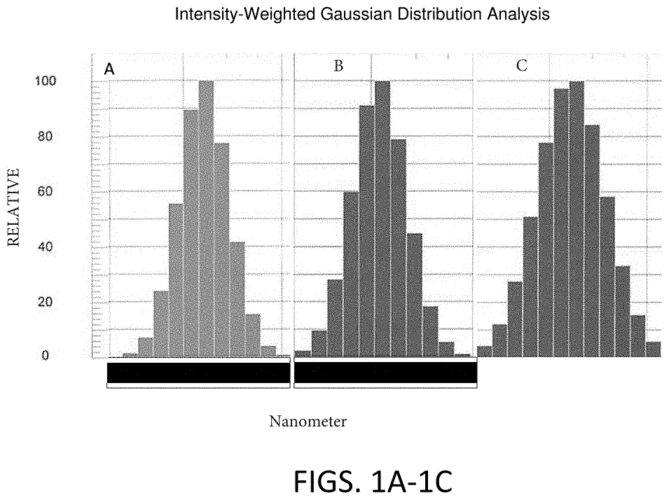

Nanoparticles, as the term is used herein, are particles having a matrix-type structure with a size of 1000 nanometers or less. The nanoparticles are generally spherical structures, but can also be other shapes. In some embodiments, the nanoparticles have a size of 500 nanometers or less. In some embodiments, the particles have a diameter from 10 nanometers to 1000 nanometers. In other embodiments, the particles have a diameter from 10 nanometers to 500 nanometers. In further embodiments, the particles have a diameter from 10 to 300 nanometers, while in yet further embodiments the particles have a diameter from 50 to 300 nanometers or 100 to 400 nanometers. The diameter of the nanoparticles refers to their mean hydrodynamic diameter. The hydrodynamic diameter can be readily determined using dynamic light scattering (DLS) (FIG. 1). In dry state, nanoparticle size can be measured by scanning electron microscopy (SEM) or transmission electron microscopy (TEM).

The nanoparticles are prepared by oil-in-water solvent evaporation method. In brief, PLGA and API (paclitaxel or sirolimus) or a combination of API are dissolve in an organic solvent (e.g., acetone, dichloromethane, dimethylformamide, eethyl acetate, or tetrahydrofuran). In addition to PLGA and API, the polymeric phase in certain embodiment contains plasticizer, fatty acid, and/or an imaging agent (Collectively called “organic phase”). The aqueous phase consists of dissolved PVA+PLL. In some embodiment, in addition to PVA+PLL, the aqueous phase contains cationic surfactant (Collectively called, “Aqueous Phase”). The organic phase is emulsified into the aqueous phase, first by sonication followed by high speed homogenization. The sonication and homogenization conditions are optimized to achieved nanoparticles in certain size range. Following evaporation of chloroform with stirring followed by under vacuum, the nanoparticle formulation is lyophilized. As a variation of the protocol, sugar is added prior to lyophilization.

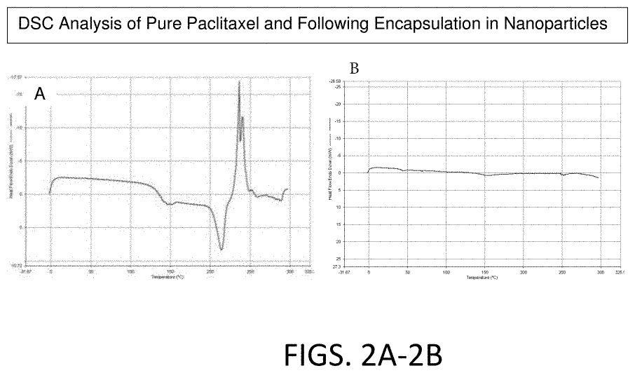

To characterize the nanoparticles, their hydrodynamic diameter was determined using dynamic light scattering (FIG. 1). In addition, zeta potential of nanoparticles was measured. To determine the nature of the encapsulated drug, nanoparticles were characterized using differential scanning calorimetry (DSC). The data were compared with pure drug (FIG. 2). The DSC results show amorphous nature of the encapsulated drug.

The pharmaceutical composition can include nanoparticles that are all essentially the same, or it can include nanoparticles having a plurality of characteristics. One characteristic that can be varied in the nanoparticles is the drug carried by the nanoparticle. For example, in some embodiments, one set of nanoparticles includes a first therapeutic agent, while a second set of nanoparticles includes a second therapeutic agent. However, additional characteristics of the nanoparticles in the coating composition can also be varied. Additional characteristics that can vary in a plurality of different types of nanoparticles included in the pharmaceutical composition include the diameter of the nanoparticles, the drug loading of the nanoparticles, the surface charge of the nanoparticles, compositions of the nanoparticle matrix, and/or the drug release profile from the nanoparticles.

### Polymers for Nanoparticles

Polymers for formulation of nanoparticles: Although the specific PLGA polymer composition and type is preferred for a single or dual active pharmaceutical ingredient (API) (e.g., the sirolimus and paclitaxel combination), there are other biodegradable and biocompatible polymers which could be used for formulating sustained release functionalized nanoparticles with further optimization of the protocol. Biodegradable polymers are a special class of polymer that breaks down after its intended purpose. These polymers are found both naturally and synthetically made, and largely consist of ester, amide, and ether functional groups. Their properties and breakdown mechanism are determined by their exact structure. These polymers are often synthesized by condensation reactions, ring opening polymerization, and metal catalysts. In general, biodegradable polymers can be grouped into two large groups based on their structure and synthesis. One of these groups is agro-polymers, or those derived from biomass. For example, polysaccharides, like starches. The other consists of biopolyesters, which are those derived from microorganisms or synthetically made from either naturally or synthetic monomers. Examples of biopolyesters include polyhydroxybutyrate and polylactic acid, polyesters, polyanhydrides, Polyurethanes and poly(ester amide)s, blend of polymers, copolymer poly(L-lactide-co-F-caprolactone). The polymer slowly degrades into smaller fragments, releasing a natural product, and there is controlled ability to release a drug. The drug slowly releases as polymer degrades. For example, polylactic acid, poly(lactic-co-glycolic) acid, and poly(caprolactone), all of which are biodegradable and used for sustained release of the encapsulated therapeutic agent.

The nanoparticles of the invention can be prepared using a wide variety of different types of polymers. Preferably, the nanoparticle comprises one or more biocompatible polymers. Examples of biocompatible polymers include natural or synthetic polymers such as polystyrene, polylactic acid, polyketal, butadiene styrene, styreneacrylic-vinyl terpolymer, polymethylmethacrylate, polyethylmethacrylate, polyalkylcyanoacrylate, styrene-maleic anhydride copolymer, polyvinyl acetate, polyvinylpyridine, polydivinylbenzene, polybutyleneterephthalate, acrylonitrile, vinylchloride-acrylates, polycaprolactone, poly(alkyl cyanoacrylates), poly(lactic-co-glycolic acid), and the like.

In further embodiments, the nanoparticle comprises one or more biodegradable polymers. Use of biodegradable polymers provides the advantages of using nanoparticles that will eventually disintegrate, which facilitates release of the active pharmaceutical ingredients and elimination of the nanoparticles in vivo. However, active pharmaceutical agents can also be released from the matrix of non-biodegradable polymers as a result of gradual efflux from channels within the polymer matrix, including those formed by soluble materials or plasticizers included in the polymer matrix.

Examples of biodegradable polymers include polylactide polymers include poly(D,L-lactide)s; poly(lactide-co-glycolide) (PLGA) copolymers; polyglycolide (PGA) and polydioxanone; caprolactone polymers; chitosan; hydroxybutyric acids; polyanhydrides and polyesters; polyphosphazenes; and polyphosphoesters. A preferred biodegradable polymer for use in the nanoparticles is poly(lactide-co-glycolide).

Modified poly(D,L-lactide)s can also be used as biodegradable polymers in the nanoparticles of the invention. Examples of modified poly(D,L-lactide)s include poly(L-lactide), acrylate terminated; poly(L-lactide), amine terminated; poly(L-lactide), azide terminated; poly(L-lactide), 2-bromoisobutyryl terminated; poly(L-lactide), 2-bromoisobutyryl terminated; poly(L-lactide) 4-cyano-4-[(dodecylsulfanylthiocarbonyl)sulfanyl]pentonate; poly(L-lactide) N-2-hydroxyethylmaleimide terminated; poly(L-lactide) 2-hydroxyethyl, methacrylate terminated; poly(L-lactide), propargyl terminated; poly(L-lactide), thiol terminated;

Other biodegradable polymers that can be used in the nanoparticles include AB diblock copolymers such as poly(ethylene glycol) methyl ether-block-poly(D,L-lactide); poly(ethylene glycol) methyl ether-block-poly(lactide-co-glycolide) PEG; poly(ethylene glycol)-block-poly(F-caprolactone) methyl ether PEG; and polypyrrole-block-poly(caprolactone). Further biodegradable polymers include ABA triblock copolymers such as polylactide-block-poly(ethylene glycol)-block-polylactide PLA; poly(lactide-co-glycolide)-block-poly(ethylene glycol)-block-poly(lactide-co-glycolide); poly(lactide-co-caprolactone)-block-poly(ethylene glycol)-block-poly(lactide-co-caprolactone); polycaprolactone-block-polytetrahydrofuran-block-polycaprolactone; and polyglycolide-block-poly(ethylene glycol)-block-polyglycolide PEG.

Another biodegradable polymer that can be used in some embodiments of the invention is an N-alkylacrylamide copolymer. N-alkylacrylamide is a hydrophobic monomer having an alkyl group of C₃ to C₆. For example, in some embodiments, the biodegradable polymer is a copolymer of an N-alkylacrylamide, a vinyl monomer, and a polyethylene glycol (PEG) conjugate. However, in some embodiments, the biodegradable polymer is any of the biodegradable polymers described herein other than a copolymer of an N-alkylacrylamide, a vinyl monomer, and a PEG conjugate. Use of nanoparticles comprising biodegradable polymers a copolymer of an N-alkylacrylamide, a vinyl monomer, and a PEG conjugate are described in U.S. Pat. No. 9,138,416, the disclosure of which is incorporated herein by reference.

Biodegradable polymers also include various natural polymers. Examples of natural polymers include polypeptides including those modified non-peptide components, such as saccharide chains and lipids; nucleotides; sugar-based biopolymers such as polysaccharides; cellulose; carbohydrates and starches; dextrans; lignins; polyamino acids; adhesion proteins; lipids and phospholipids (e.g., phosphorylcholine).

### Functionalized Nanoparticles

In some embodiments, the polymeric nanoparticles are functionalized, or “surface-functionalized.” Surface functionalization is a method of altering the surface properties of a material to achieve specific goals such as inducing a desired bioresponse or inhibiting a potentially adverse reaction. Functionalized nanoparticles are described in U.S. Pat. No. 8,865,216, the disclosure of which is incorporated herein by reference.

In some embodiments, functionalized nanoparticles comprise a biocompatible polymer having a net positive or negative charge at neutral pH, at least one charge modulator that is effective to reverse the surface charge from negative to positive in an acidic environment, and optionally an amphiphilic emulsifier. For example, in some embodiments, the polymeric nanoparticle has a cationic zeta potential. Functionalized nanoparticles are prepared using the combination of poly vinyl alcohol (or other amphiphilic emulsifier) with poly-L-lysine (or other charge modulator) are referred to herein as modified or surface modified nanoparticles. Generally speaking, surface modification of nanoparticles, as described herein, produces a significant increase in the cellular uptake, as compared to unmodified nanoparticles. In some embodiments, the polymeric nanoparticles are surface-functionalized with PVA and poly-1-lysine (PLL), while in additional embodiments the polymeric nanoparticles are further surface-functionalized with a cationic surfactant.

In some embodiments, the functionalized polymeric nanoparticles include PVA, PLL, and DDAB, and their different combinations for preparation of functionalized nanoparticles. However, there are other agents and combination their off can also be used. Surface functionalizing agent (s) anchors at the nanoparticle surface, and thus imparts changes to surface characteristics of the nanoparticles. Agents for surface modulation to make functionalized nanoparticles include surfactants, emulsifier, peptides, proteins, lipids, vitamins, steroids, fatty acids, polymers, cell adhesion molecules, either alone or in combination to achieve surface properties to facilitate cellular/tissue uptake and retention and hence that of the encapsulated/adsorbed/conjugated therapeutic agent(s), when coated onto medical devices.

Examples of cationic surfactants for developing functionalized nanoparticles include Si-C16-Trimethylhexadecylammonium bromide (CTAB), Si-C18-Trimethyloctadecylammonium bromide (TOAB), Di-C12-Dimethyldodecylammonium bromide (DDAB), Di-C16-Dihexadecyldimethylammonium bromide (DHDB), Behentrimonium chloride, Benzalkonium chloride, Benzethonium chloride, Benzododecinium bromide, Bronidox, Carbethopendecinium bromide, Cetalkonium chloride, Cetrimonium bromide, Cetrimonium chloride, Cetylpyridinium chloride, Lauryl methyl gluceth-10 hydroxypropyl dimonium chloride, Octenidine dihydrochloride, Olaflur, N-Oleyl-1,3-propanediamine, Stearalkonium chloride, Tetramethylammonium hydroxide, and Thonzonium bromide.

Zwitterionic (amphoteric) surfactants have both cationic and anionic centers attached to the same molecule. The cationic part is based on primary, secondary, or tertiary amines or quaternary ammonium cations. The anionic part can be more variable and include sulfonates, as in the sultaines CHAPS (3-[(3-cholamidopropyl)dimethylammonio]-1-propanesulfonate) and cocamidopropyl hydroxysultaine. Betaines such as cocamidopropyl betaine have a carboxylate with the ammonium. The most common biological zwitterionic surfactants have a phosphate anion with an amine or ammonium, such as the phospholipids phosphatidylserine, phosphatidylethanolamine, phosphatidylcholine, and sphingomyelins.

Anionic surfactants contain anionic functional groups at their head, such as sulfate, sulfonate, phosphate, and carboxylates. Prominent alkyl sulfates include ammonium lauryl sulfate, sodium lauryl sulfate (sodium dodecyl sulfate, SLS, or SDS), and the related alkyl-ether sulfates sodium laureth sulfate (sodium lauryl ether sulfate or SLES), and sodium myreth sulfate. Other examples of anionic surfactants include Docusate (dioctyl sodium sulfosuccinate), perfluorooctanesulfonate, perfluorobutanesulfonate, alkyl-aryl ether phosphates, and alkyl ether phosphates.

Carboxylates are the most common surfactants and comprise the carboxylate salts (soaps), such as sodium stearate. More specialized species include sodium lauroyl sarcosinate and carboxylate-based fluorosurfactants such as perfluorononanoate or perfluorooctanoate.

In some embodiments, the polymeric nanoparticles are functionalized using non-ionic surfactants. Non-ionic surfactants have covalently bonded oxygen-containing hydrophilic groups, which are bonded to hydrophobic parent structures. The water-solubility of the oxygen groups is the result of hydrogen bonding. Hydrogen bonding decreases with increasing temperature, and the water solubility of non-ionic surfactants therefore decreases with increasing temperature.

Examples of non-ionic surfactants include ethoxylates, fatty alcohol ethoxylates, narrow-range ethoxylate, octaethylene glycol monododecyl ether, pentaethylene glycol monododecyl ether, alkylphenol ethoxylates (APEs or APEOs), nonoxynols, triton X-100, and fatty acid ethoxylates. Fatty acid ethoxylates are a class of very versatile surfactants, which combine in a single molecule the characteristic of a weakly anionic, pH-responsive head group with the presence of stabilizing and temperature responsive ethylene oxide units.

Additional examples of non-ionic surfactants include special ethoxylated fatty esters and oils, ethoxylated amines and/or fatty acid amides, polyethoxylated tallow amine, cocamide monoethanolamine, cocamide diethanolamine, terminally blocked ethoxylates, poloxamers, fatty acid esters of polyhydroxy compounds, fatty acid esters of glycerol, glycerol monostearate, glycerol monolaurate, fatty acid esters of sorbitol, Spans such as sorbitan monolaurate, sorbitan monostearate, and sorbitan tristearate, tweens, such as tween 20, tween 40, tween 60, and tween 80, fatty acid esters of sucrose, alkyl polyglucosides such as decyl glucoside, lauryl glucoside, and octyl glucoside, amine oxides such as lauryldimethylamine oxide.

Examples of natural surfactants for developing functionalized nanoparticles include surface-active lipoprotein complex (phospholipoprotein), dipalmitoylphosphatidylcholine (DPPC), phosphatidylcholine, and apolipoproteins.

Surfactants can be used in combination or alone to modulate nanoparticle surface properties. In addition, they could be used in combination with polymeric surfactants, and various types of polymers (e.g., anionic, cationic, neutral, amphoteric). Polymeric surfactants or with functional groups (carbonylated, caroboxylated, amine and amide modified, etc.). Surfactants can help in changing the permeability of the tissue to facilitate uptake of nanoparticles.

In some embodiments, the polymeric nanoparticles are functionalized using cationic, anionic, neutral, or amphoteric polymers.

Examples of cationic polymers include poly(ethyleneimine) (PEI), poly-1-(lysine) (PLL), poly[2-(N,N-dimethylamino)ethyl methacrylate] (PDMAEMA) and chitosan, DEAE dextran, (vinylpyrrolidone-N and N-dimethylaminoethyl methacrylate acid copolymer diethyl sulphate, cationic gelatin, cationic cellulose, poly(2-N,N-dimethylaminoethylmethacrylate) (PDMAEMA), Poly-1-lysine (PLL), poly(amidoamine) (PAMAM), diallyldimethyl ammonium chloride (DADMAC), and polyamide-amines.

Examples of neutral polymers include polyethylene oxides (PEO), polyvinyl alcohol (PVA), and polyethylene glycol. Examples of anionic polymers include polyacrylamide, poly(N-isopropyl acrylamide), poly(N-octyl acrylamide), poly(N-tert-butyl acrylamide), poly(N-phenyl acrylamide), poly(N-sec-butyl acrylamide)polystyrene sulphonate, and sodium polyvinyl sulphonate. Examples of amphoteric polymers include acrylamide-acrylic acid-DMAEA-MeCl or DADMAC-acrylic acid.

### Therapeutic Agents

The polymeric nanoparticles include a first therapeutic agent and a second therapeutic agent. Nanoparticles with combination of drugs/two different formulations of nanoparticles can be incorporated, if there is synergistic effect. Alternatively, the therapeutic agents can be localized in different layers of polymeric matrix. For e.g., outside layer can contain nanoparticles with antiproliferative drug whereas the inside layer includes nanoparticles including endothelial nitric oxide synthase to promote endothelialization.

The first and second therapeutic agents can be included in the nanoparticles in various different ways. For example, the therapeutic agents can be dispersed within the nanoparticle, or it can be encapsulated within the nanoparticle. In some embodiments, the therapeutic agents are substantially evenly dispersed within the nanoparticle. Alternately, one or more of the therapeutic agents can be conjugated to a nanoparticle surface. Likewise, a combination of the surface associated therapeutic agent and therapeutic agent dispersed in polymeric nanoparticle can be used.

When included in the polymeric nanoparticles, the first and second therapeutic agents can both be included in the same nanoparticles, or they can be segregated into different nanoparticles. For example, in some embodiments, the sirolimus and the paclitaxel are present in different polymeric nanoparticles, while in other embodiments the sirolimus and the paclitaxel are present within the same polymeric nanoparticles.

The first and second therapeutic agents can be included in the pharmaceutical composition and the nanoparticles in a variety of different ratios. For example, the weight ratio of the first therapeutic agent to the second therapeutic agent can range from about 1:10 to about 10:1, from 1:8 to about 8:1, from about 1:5 to about 5:1, from about 1:3 to about 3:1, from about 2:1 to about 1:2, from about 3:2 to about 2:3, or about 1:1 (all by weight). For example, in some embodiments, the weight ratio of sirolimus to paclitaxel ranges from about 1:2 w/w to about 2:1 w/w.

In some embodiments, it is preferable to include a higher amount of one of the therapeutic agents. For example, the inventors have determined that when using sirolimus and paclitaxel, different effects can be obtained depending on the ratio of the two therapeutic agents, with a high relative amount of sirolimus resulting in a cytostatic effect, while a high relative amount of paclitaxel results in a cytotoxic effect. For example, the weight ratio of the first therapeutic agent to the second therapeutic agent can range from about 2:1 to about 10:1, from about 3:1 to about 10:1, from about 5:1 to about 10:1, from about 8:1 to about 10:1, or from about 9:1 to about 10:1 (all by weight). For example, in some embodiments the weight ratio of paclitaxel to sirolimus ranges from about 8:1 w/w to about 10:1 w/w, while in another embodiment the weight ratio of sirolimus to paclitaxel ranges from about 8:1 w/w to about 10:1 w/w.

The polymeric nanoparticles are limited in the amount of therapeutic agent they can include, which is referred to as their drug loading capacity. In some embodiments, the polymeric nanoparticles have a drug loading capacity of about 15% by weight. In other embodiments, the polymeric nanoparticles have a drug loading capacity from about 10% to about 15%, from about 5% to about 15%, or from about 5% to about 10%.

The pharmaceutical compositions include a first and a second therapeutic agent. The first and second therapeutic agents are different compounds. A wide variety of therapeutic agents can be included as first and second therapeutic agents. In addition, in some embodiments, additional therapeutic agents beyond the first and second therapeutic agents are included.

The formulation of nanoparticles could contain drugs (API) with different pharmacological action such as antiproliferative agents, cytotoxic, cytostatic, tissue healing, agents promoting re-reendothelialization, anti-inflammatory agents, steroids, growth factors, antibacterial, antibiotics, antioxidants, etc. Note that some drugs have multiple effects, and therefore may be listed under multiple categories for different pharmacological actions. The therapeutic agents can be small molecule or macromolecules, used in combination encapsulated in a single nanoparticle or made into different nanoparticle formulations, used in different ratio in combination, with different release profile, drug load, nanoparticles of different sizes and physical properties such as surface charge (zeta potential), dissolution profile, etc.

In some embodiments, the first and/or the second therapeutic agent can be an antiproliferative drug. One category of antiproliferative drugs are limus drugs. Limus drugs are cytostatic, rather than cytotoxic, which means that the cell becomes inhibited from proliferation if therapeutic tissue levels are maintained over time. Examples of limus drugs include sirolimus, zotarolimus, and everolimus.

One important clinical difference between limuses, such as sirolimus, and paclitaxel/taxol derivatives is the fact that sirolimus degrades relatively quickly once it is put into solution. So, once sirolimus is in the tissue, it remains bioactive for a few days, which requires the drug to be protected following drug release. On the other hand, paclitaxel is relatively stable drug, particularly in crystalline form; it can be delivered into the tissue and stays there for a long time. The challenge with limus derivatives is the need for control-release mechanisms. Also, due to paclitaxel's crystalline nature, it can penetrate the tissue as the balloon is inflated whereas limus drugs due to their amorphous nature do not diffuse well.

Antimetabolites or cytotoxic drugs can also be used as antiproliferative agents. Examples include paclitaxel or taxol family (i.e., taxanes), Mycophenolate Mofetil, Mycophenolate Sodium and Azathioprine, anti-folates, fluoropyrimidines, deoxynucleoside analogues and thiopurines. Example, methotrexate and pemetrexed. Methotrexate inhibits dihydrofolate reductase (DHFR), an enzyme that regenerates tetrahydrofolate from dihydrofolate. Taxane compounds are a class of diterpene compounds, and include paclitaxel, docetaxel, abeotaxane, and taxine.

Anti-microtubule agents are antiproliferative plant-derived chemicals that block cell division by preventing microtubule function. Microtubules are an important cellular structure composed of two proteins; α-tubulin and β-tubulin. They are hollow, rod-shaped structures that are required for cell division, among other cellular functions. Microtubules are dynamic structures, which means that they are permanently in a state of assembly and disassembly. Vinca alkaloids and taxanes are the two main groups of anti-microtubule agents, and although both of these groups of drugs cause microtubule dysfunction, their mechanisms of action are completely opposite. The vinca alkaloids prevent the formation of the microtubules, whereas the taxanes prevent the microtubule disassembly. By doing so, they prevent the cancer cells from completing mitosis. Following this, cell cycle arrest occurs, which induces programmed cell death (apoptosis). Docetaxel, semi-synthetically prepared. Paclitaxel prevents the cell cycle at the boundary of G2-M, whereas docetaxel exerts its effect during S-phase.

In some embodiments, one or both of the therapeutic agents are topoisomerase inhibitors. Examples of topoisomerase inhibitors include irinotecan and topotecan, are semi-synthetically derived from camptothecin, etoposide, doxorubicin, mitoxantrone and teniposide.

In some embodiments, the first and/or the second therapeutic agent can be a cytotoxic antibiotic compound. Examples of cytotoxic antibiotics include anthracyclines and the bleomycins; other prominent examples include mitomycin C and actinomycin. Among the anthracyclines, doxorubicin and daunorubicin were the first, and were obtained from the bacterium Streptomyces peucetius. Derivatives of these compounds include epirubicin and idarubicin. Other clinically used drugs in the anthracycline group are pirarubicin, aclarubicin, and mitoxantrone.

In some embodiments, the first and/or the second therapeutic agent can be an anti-inflammatory compound. Representative examples of such agents include nonsteroidal agents (NSAIDS) such as salicylates, diclofenac, diflunisal, flurbiprofen, ibuprofen, indomethacin, mefenamic acid, nabumetone, naproxen, piroxicam, ketoprofen, ketorolac, sulindac, tolmetin. Other anti-inflammatory drugs include steroidal agents such as beclomethasone, betamethasone, cortisone, dexamethasone, fluocinolone, flunisolide, hydorcortisone, prednisolone, and prednisone. Immunosuppressive agents are also contemplated (e.g., adenocorticosteroids, cyclosporin).

In some embodiments, the first and/or the second therapeutic agent can be a cardiovascular receptor modulating compound. Examples include adrenergic blockers and stimulators (e.g., doxazosin, guanadrel, guanethidine, pheoxybenzamine, terazosin, clonidine, guanabenz); alpha-/beta-adrenergic blockers (e.g., labetalol); angiotensin converting enzyme (ACE) inhibitors (e.g., benazepril, catopril, lisinopril, ramipril); ACE-receptor antagonists (e.g., losartan); beta blockers (e.g., acebutolol, atenolol, carteolol, pindolol, propranolol, penbatolol, nadolol); and calcium channel blockers (e.g., amiloride, bepridil, nifedipine, verapamil, nimodipine).

In some embodiments, the first and/or the second therapeutic agent can include other cardiovascular agents such as antiarrythmics, groups I-IV (e.g., bretylium, lidocaine, mexiletine, quinidine, propranolol, verapamil, diltiazem, trichlormethiazide, metoprolol tartrate, carteolol hydrochloride); and miscellaneous antiarrythmics and cardiotonics (e.g., adenosine, digoxin, caffeine, dopamine hydrochloride, digitalis).

In some embodiments, the first and/or the second therapeutic agent can include a growth factor. Growth factors typically promote cell differentiation and maturation, which varies between growth factors. For example, epidermal growth factor (EGF) enhances osteogenic differentiation, while fibroblast growth factors and vascular endothelial growth factors stimulate blood vessel differentiation (angiogenesis). Erythropoietin (EPO), Fetal Bovine Somatotrophin (FBS), Insulin-like growth factors, Platelet derived growth factor, Keratinocyte growth factor. Growth factors such as VEGF, stromal cell-derived factor-1 (SDF-1), and granulocyte colony-stimulating factor (G-CSF) have been found to increase mobilization of EPCs from the bone marrow and used in vascular graft applications. CD31 antibodies has been shown to induce EC-specific binding, promoting attachment and long-term adhesion of ECs. Anti-CD34 antibodies have been used in a variety of applications to aid in the endothelialization of vascular stents. Heparin-modified substrates could prevent platelet adhesion and inhibit the growth and suppress the proliferation of SMCs.

In some embodiments, the first and/or the second therapeutic agent can be an anti-restenotic agent, or anti-apoptic agent. Examples of anti-restenotic agent include rapamycin (i.e., sirolimus) or a derivative or analog thereof, e.g., everolimus or tacrolimus. Examples of anti-apoptic agents include Galectin-3; (−)deprenyl; monoamine oxidase inhibitors (MAO-I) such as selegiline and rasagiline; rapamycin; or quercetin; paclitaxel.

Other suitable first and second therapeutic agents include agents that inhibit tissue damage. Representative examples of such agents include antioxidants such as superoxide dismutase, catalase, glutathione, Vitamin E; immune modulators (e.g., lymphokines, monokines, interferon α and γ); and growth regulators (e.g., IL-2, tumor necrosis factor, epithelial growth factor, vascular endothelial growth factor, fibroblast growth factor, transforming growth factor-beta, somatrem, fibronectin, GM-CSF, CSF, platelet-derived growth factor, somatotropin, rG-CSF, epidermal growth factor, IGF-1).

In some embodiments, the first and/or the second therapeutic agent can be an agent to accelerate re-endothelialization. Several molecules have been used to improve the cell-material interactions in order to enhance the adhesion and growth of ECs and consequently accelerate re-re-endothelialization, for example, heparin, fucoidan, chondroitin sulfate, hyaluronic acid, antioxidant compounds, and extracellular matrix (ECM) proteins including laminin, fibronectin, and collagen. Nitric oxide (NO) releasing enzymes (eNOs) or compounds that produces nitric oxide. These could be genes or proteins.

In some embodiments, the first and/or the second therapeutic agent can be an antifibrotic agents. These agents reduce fibroblast proliferation that may decrease the formation and/or accumulation of fibrotic materials within the tissue. These agents when delivered to the blood vessels could reduce fibrous tissue formation which could help in the healing process of the injured artery. Example, Nintedanib and Pirfenidon.

In some embodiments, the first and/or the second therapeutic agent can be a denaturing agent. Examples of denaturing agents include guanidinium chloride, urea, sodium salicylate, propylene to disrupt network of proteins to facilitate the diffusion of nanoparticles through the arterial wall. Agents that break disulfide bonds such as mercaptoethanol, Dithiothreitol, TCEP (tris(2-carboxyethyl)phosphine).

In some embodiments, the first and/or the second therapeutic agent can be an agent to remove calcium deposits in blood vessels. Hydroxyapatite is a type of calcium phosphate. Corticosteroids, such as triamcinolone acetonide and triamcinolone diacetate, calcium channel blockers, such as amlodipine (Norvasc), diltiazem (Cardizem, Tiazac) and verapamil (Calan, Verelan), colchicine (Colcrys), an anti-inflammatory medication. Drugs such as interferon beta-1a, nadroparin and para-amino-salicylic acid could also be used. Calcium chelating agents such as Edetic acid, Citric acid, EDTA. Removal of calcium deposits could help in the healing process.

In some embodiments, the first and/or the second therapeutic agent can be an antioxidant. Examples of antioxidants include antioxidant enzymes (e.g., superoxide dismutase, catalase, Ubiquinol, peroxiredoxins) or compounds (e.g., selenium or vitamin E, Lipoic acid, Uric acid) that can neutralize reactive oxygen species (ROS). ROS activate cell proliferation and are functionally involved in the development of arterial stenosis after balloon angioplasty.

The first and second therapeutic agents can be provided in various forms. For example, the therapeutic agents can be provided in amorphous or crystalline forms. Crystalline solids can exist in several subphases, such as polymorphs, solvates, hydrates, and co-crystals. The nature of the crystalline form of a drug substance may affect its stability in the solid state, its solution properties, and its absorption. In some embodiments, one of the therapeutic agents is amorphous, while the other therapeutic agent is crystalline. In some embodiments, the sirolimus and/or the paclitaxel is amorphous.

### Cell Penetrating Peptides

In some embodiments, the nanoparticles include cell-penetrating peptides. Cell-penetrating peptides (CPPs) are short peptides that facilitate cellular intake/uptake of various molecular equipment (from nanosize particles to small chemical molecules and large fragments of DNA). The “cargo” is associated with the peptides either through chemical linkage via covalent bonds or through non-covalent interactions. The function of the CPPs is to deliver the cargo into cells, a process that commonly occurs through endocytosis with the cargo delivered to delivery vectors for use in research and medicine.

CPPs typically have an amino acid composition that either contains a high relative abundance of positively charged amino acids such as lysine or arginine or has sequences that contain an alternating pattern of polar/charged amino acids and non-polar, hydrophobic amino acids. These two types of structures are referred to as polycationic or amphipathic, respectively. A third class of CPPs are the hydrophobic peptides, containing only apolar residues, with low net charge or have hydrophobic amino acid groups that are crucial for cellular uptake.

An example of direct penetration has been proposed for TAT. The first step in this proposed model is an interaction with the unfolded fusion protein (TAT) and the membrane through electrostatic interactions, which disrupt the membrane enough to allow the fusion protein to cross the membrane. After internalization, the fusion protein refolds due the chaperone system. The mechanism of action of CCPs involves strong interactions between cell-penetrating peptides and the phosphate groups on both sides of the lipid bilayer, the insertion of positively charged arginine side-chains that nucleate the formation of a transient pore, followed by the translocation of cell-penetrating peptides by diffusing on the pore surface.

Based on the origin of peptides, CPPs are divided into chimeric, protein-derived and synthetic. Chimeric CPPs are composed of two or more motifs from dissimilar peptides. Transportan is a chimeric CPP, which derived from galanin and mastoparan. TAT and penetratin that derived from natural proteins are examples of protein-derived CPPs. The polyarginine family, the simplest CPP mimics with arginine as the only structural component, belongs to synthetic peptides.

Amphipathic CPPs are the sequences with a high degree of amphipathicity because of lysine residues in their structures. Transportan (a 27 amino acid-long peptide) is an amphipathic CPP. Hydrophobic CPPs contain only hydrophobic motif/non-polar sequences. Generally, there are a few reports for the use of these CPPs as carriers compared to cationic and amphipathic CPPs.

In some embodiments, the nanoparticle includes one or more fatty acids and lipids. Fatty acids include saturated, monounsaturated, polyunsaturated fatty acids. Specific examples of fatty acids include omega-3, alpha-linolenic acid, steric acid, eicosapentaenoic and docosahexaenoic acid, omega-6, arachidonic acid, linoleic acid, conjugated linoleic acid, trans fatty acids, short-chain fatty acids, alpha-lipoic acid, medium-chain fatty acids, long-chain fatty acids, very long-chain fatty acids, monoglycerides, diglycerides, triglycerides, phospholipids, lecithin (phosphatidylcholine), sterols such as cholesterol and phytosterols, steroids such as estrogen, and testosterone. Other examples of lipids include bile salts, cortisol, fat-soluble vitamins such as vitamin A, vitamin D, vitamin E, and vitamin K.

### Supplemental Materials

The nanoparticles of the invention can include other compounds in addition to the first and second therapeutic agents. For example, the nanoparticles can include an additional protein such as albumin within the nanoparticle. The presence of an additional protein (e.g., albumin) can be useful for facilitating release of a therapeutic agent from the nanoparticle by acting as a bulking agent. The presence of an additional protein (e.g., albumin) can also serve to protect the therapeutic agent from interfacial inactivation by contact with the organic/aqueous interface during preparation of the nanoparticles.

Polymeric nanoparticles can also include plasticizers to modulate the release of the encapsulated drug(s) and improve elasticity. The wide variety of ester chemistries that are in production include sebacates, adipates, terephthalates, dibenzoates, gluterates, phthalates, azelates, tartrates, and other specialty blends. A wide range of elastomers including nitrile, polychloroprene, EPDM, chlorinated polyethylene, and epichlorohydrin. Plasticizers based on esters of polycarboxylic acids with linear or branched aliphatic alcohols of moderate chain length. Phthalate esters of straight-chain and branched-chain alkyl alcohols. Ortho-phthalate esters. Superplasticizers have generally been manufactured from sulfonated naphthalene condensate or sulfonated melamine formaldehyde, although newer products based on polycarboxylic ethers. Lignin, naphthalene, and melamine sulfonate superplasticisers are organic polymers. Polycarboxylate ether superplasticizer or just polycarboxylate, work differently from sulfonate-based superplasticizers, giving steric stabilization, instead of electrostatic repulsion.

Additional examples of plasticizers include trimellitates, trimethyl trimellitate, tri-(2-ethylhexyl) trimellitate, tri-(n-octyl,n-decyl) trimellitate, Tri-(heptyl,nonyl) trimellitate, n-octyl trimellitate, adipates, sebacates, maleates. bis(2-ethylhexyl)adipate, dimethyl adipate, monomethyl adipate, dioctyl adipate, dibutyl sebacate, dibutyl maleate, diisobutyl maleate, azelates, benzoates, Terephthalates such as dioctyl terephthalate/DEHT, 1,2-Cyclohexane dicarboxylic acid diisononyl ester, alkyl sulphonic acid phenyl ester, sulfonamides: N-ethyl toluene sulfonamide, N-(2-hydroxypropyl) benzene sulfonamide, N-(n-butyl) benzene sulfonamide, organophosphates such as tricresyl phosphate, tributyl phosphate, glycols and polyethers such as triethylene glycol dihexanoate, tetraethylene glycol diheptanoate, polymeric plasticizers such as olybutene, bio-based plasticizers such as acetylated monoglycerides, alkyl citrates such as triethyl citrate, acetyl triethyl citrate, tributyl citrate, acetyl tributyl citrate, trioctyl citrate, acetyl trioctyl citrate, trihexyl citrate, acetyl trihexyl citrate, butyryl trihexyl citrate, trimethyl citrate, methyl ricinoleate, and green plasticizers such as epoxidized soybean oil and epoxidized vegetable oils.

Other plasticizers include nitroglycerine (NG, aka “nitro”, glyceryl trinitrate), butanetriol trinitrate, dinitrotoluene, trimethylolethane trinitrate, diethylene glycol dinitrate, triethylene glycol dinitrate, bis(2,2-dinitropropyl)formal, bis(2,2-dinitropropyl)acetal, and 2,2,2-Trinitroethyl 2-nitroxyethyl ether.

In some embodiments, the polymer nanoparticles further comprise a solubilizing agent. An example of a preferred solubilizing agent is HS-15 ((15)-hydroxystearate). Additional solubilizing agents include macrogolglycerol hydroxystearate, sodium laurylsulfate, poloxamer 188, povidone, polysorbates, and triacetin. Solubilizing agents can also be included in the polymer matrix. Solubilized therapeutic agents can provide higher tissue update.

In some embodiments, the polymeric nanoparticles further comprise L-(+)-tartaric acid dimethyl ester or a fatty acid. Dimethyltartaric acid added into nanoparticles that facilitates the release of encapsulated drug. Dimethyl tartaric acid in nanoparticles can improve the ability to modulate drug loading and release rate from the polymeric nanoparticles.

In addition to therapeutic agent, nanoparticles or coating material further comprise an imaging agent. Examples of imaging agents include dyes (visual, Near-infrared red) for optical imaging, characterization of coating material and release of nanoparticles. Contrast agents (e.g., for MRI, microCT) or radiolabeled agents to monitor transport to the tissue are other types of imaging agents.

Specific examples of imaging agents include gadoterate (Dotarem, Clariscan), gadodiamide (Omniscan), gadobenate (MultiHance), gadopentetate (Magnevist), gadoteridol (ProHance), gadoversetamide (OptiMARK), gadobutrol (Gadovist [EU]/Gadavist [US]), gadopentetic acid dimeglumine (Magnetol), blood pool agents, albumin-binding gadolinium complexes, gadofosveset (Ablavar, formerly Vasovist), gadocoletic acid, polymeric gadolinium complexes, gadomelitol, gadomer, hepatobiliary (liver) agents, gadoxetic acid, iron platinum: superparamagnetic, manganese: paramagnetic, protein-based MRI contrast agents, enzyme-activated MR contrast agents, and radioisotopes of hydrogen, carbon, phosphorus, sulfur, and iodine.

### Coating Compositions

The pharmaceutical composition includes a polymeric coating composition comprising polymeric nanoparticles dispersed within a polymeric matrix. Including the polymeric nanoparticles provides one or more advantages. The polymeric matrix can adhere the polymeric nanoparticles to an implantable medical device and can help control the rate of release of the first and second therapeutic agents included in the polymeric nanoparticles. The polymeric matrix typically includes multiple layers, based on how the pharmaceutical composition is prepared. For example, the pharmaceutical composition can include a polymer matrix with nanoparticles that can be wrapped around balloon in multiple layers followed by coating an implantable medical device with a plain polymer solution.

In some embodiments, the polymer matrix is an adhesive polymer matrix. Adhesive polymers include synthetic adhesive polymers, and bioadhesives. Bioadhesives are natural polymeric materials that act as adhesives. Bioadhesives may consist of a variety of substances, but proteins and carbohydrates feature prominently. Proteins such as gelatin and carbohydrates such as starch have been used as general-purpose glues. For example, bioadhesives secreted by microbes and by marine mollusks and crustaceans.

Bioadhesive polymers include natural polymers such as protein-based polymers, collagen, albumin, and gelatin. Polysaccharides Agarose, alginate, carrageenan, hyaluronic acid, dextran, chitosan, and cyclodextrins can also function as bioadhesive polymers.

In some embodiments, the polymeric matrix comprises a synthetic polymer. Synthetic polymers include biodegradable polyesters, poly(lactic acid), poly(glycolic acid), poly(hydroxy butyrate), poly(εcaprolactone), poly(β-malic acid), poly(dioxanones), polyanhydrides, poly(sebacic acid), poly(adipic acid), poly(terphthalic acid), polyamides, poly(imino carbonates), and polyamino acids.

Additional examples of synthetic polymers that can be used to form the polymeric matrix include phosphorous-based polymers polyphosphates, polyphosphonates, polyphosphazene, poly(cyano acrylates), polyurethanes, polyortho esters, polydihydropyrans, polyacetals, non-biodegradable cellulose derivatives, carboxymethyl cellulose, ethyl cellulose, cellulose acetate, cellulose acetate propionate, hydroxypropyl methyl, cellulose Silicones Polydimethylsiloxane, colloidal silica, acrylic polymers such as polymethacrylates, poly(methyl methacrylate), poly hydro(ethyl-methacrylate), and other polymers such as polyvinyl pyrrolidone, ethyl vinyl acetate, poloxamers, and poloxamines.

Polymeric blends can be composed by poloxamer 407 (P407) and carbomers (Carbopol 934P, Carbopol 971P, Carbopol 974P), polycarbophil, chitosan, alginates, guar gum, carrageenan, and polymers derived from cellulose can also be used to form the polymeric matrix.

A preferred adhesive polymer for use in forming the polymeric matrix is polyvinyl alcohol (PVA), which has the formula [CH₂CH(OH)]ₙ. The adhesive nature of polyvinyl alcohol that can facilitate adherence of the nanoparticles to an implantable medical device, and delivery and retention of nanoparticles in the arterial wall. Examples of other similar polymers that can be used to form the polymer matrix include polyvinyl acetate, vinyl acetate, and polyvinyl nitrate.

The polymer coating composition comprising the polymer matrix and the polymeric nanoparticles can be lyophilized, stored, and reconstituted in water or buffer or solvent composition for redispersion and coating onto medical devices.

### Coated Medical Devices

Another aspect of the present invention provides a coated implantable medical device, wherein at least a portion of the implantable medical device is coated with pharmaceutical composition, comprising a polymeric coating composition comprising polymeric nanoparticles dispersed within a polymeric matrix, wherein the polymeric nanoparticles include a first therapeutic agent and a second therapeutic agent. The polymeric nanoparticle and the polymeric matrix can include any of the compounds, and use any of the polymers, described herein. In some embodiments, the implantable or deliverable medical device is configured to fit within a blood vessel.

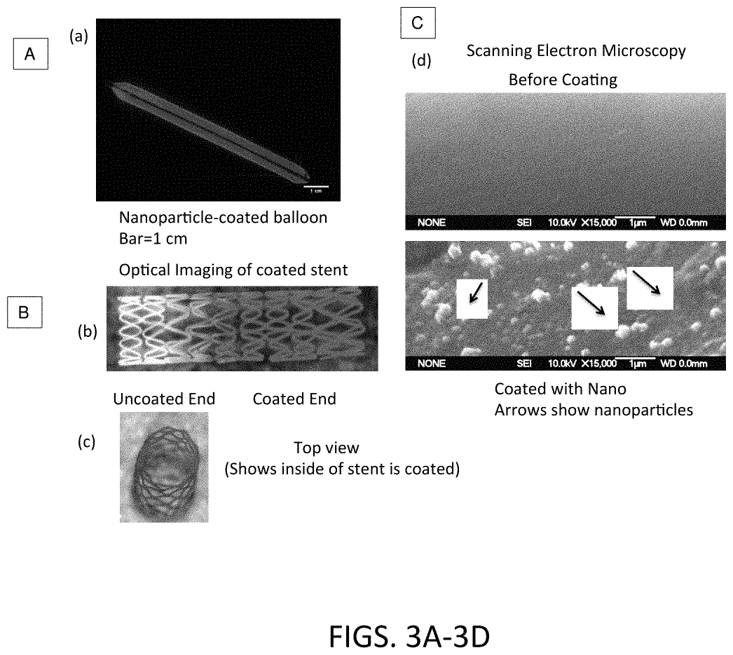

A wide variety of medical devices are known to those skilled in the art that can be coated with the pharmaceutical composition described herein. See FIG. 3. Examples of medical devices include stents, drug eluting stents; balloons, double balloon catheters; infusion catheters; vascular grafts (synthetic or biological); implanted blood access devices for hemodialysis; percutaneous medical devices; guide wires; artificial hearts, heart valves, venous valves, shunts), endovascular coils used in aneurism; pacemaker leads; intravascular and cardiovascular mesh; blood filters; transducers; blood tubing connectors; sutures; clips for coronary artery bypass graft; fistula; implantable cardioverter defibrillators; metal screws, pins, plates, and rods; intravenous catheter cannula; guide catheters; implantable electrodes, sensors, cardiac/cerebral/pulmonary shunts; pouches; wound closure dressing/glue; occluder balloons, and intravenous extension tubing. In some embodiments, the nanoparticles are used to coat non-implantable medical devices such as surgical tools. Preferred examples of implantable medical devices include vascular grafts, stents, and balloon catheters.

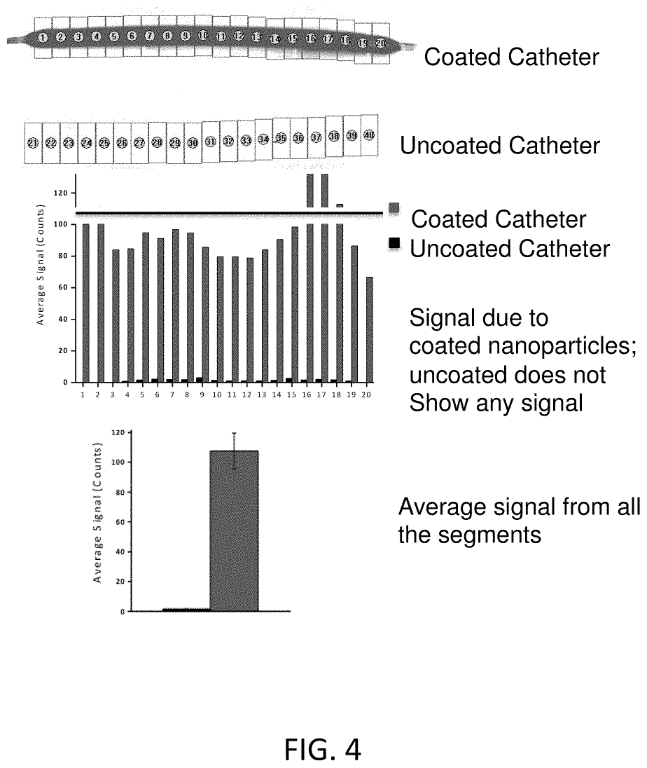

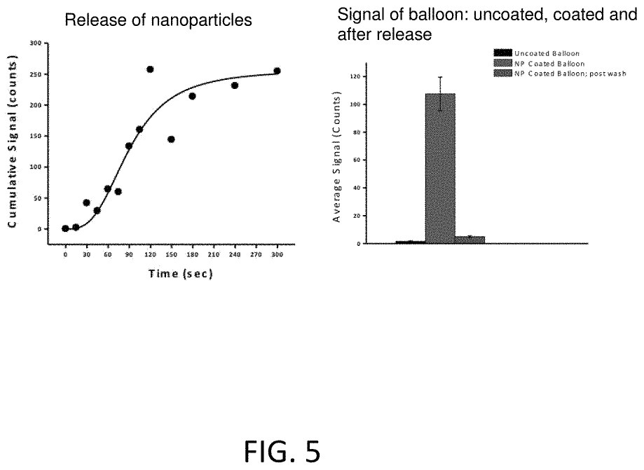

The imaging agent incorporated in nanoparticles allows determination of coating consistency across the device and the amount of nanoparticle coated onto the device (FIG. 4). In addition, the imaging agent allows to determine elution profile of the coated nanoparticles from a device under physiological conditions (FIG. 5).

The medical devices include a wide variety of biocompatible medical materials. For example, the medical devices can include biocompatible ceramics such as aluminum oxide, calcium oxide, hydroxyapatite, and zirconium(IV) oxide. The medical devices can also include biocompatible metals such as titanium or stainless steel. A wide variety of biocompatible polymers can also be used. Examples of biocompatible polymers include polyacrylics, polyamides, polyimides, polycarbonates, polydienes, polyesters, polyethers, polyfluorocarbons, polyolefins, polystyrenes, poly vinyl acetals, polyvinyl and vinylidene chlorides, polyvinyl esters, polyvinyl ethers and ketones, polyvinylpyridine, and polyvinypyrrolidone polymers.

In some embodiments, the coated implantable medical device includes an outer coating with polymer to minimize the washout effect. The outer coating is a final coating placed over the pharmaceutical composition used to coat the implantable medical device. The outer coating can include any of the polymers described for use in the polymer matrix, but in a preferred embodiment, the pharmaceutical composition is coated with an outer layer of polyvinyl alcohol.

In some embodiments, the outer coating layer can further comprise a sugar and/or glycerin to facilitate dissolution of the coating. In some embodiments, the outer layer comprises both a sugar and glycerin. In some embodiments, the sugar is glucose. Added sugar prevent aggregation of nanoparticles following coating whereas glycerin acts a plasticizer and gives elasticity to the coating, prevents flaking upon bending. This is critical when then balloon is navigated through tubes in the packaging or blood vessels with complex architecture. Glycerin also acts as a lubricant. Adding sugar along with PVA with dispersed nanoparticles also increases the adhesiveness of the polymeric coating matrix to an implantable medical device.

The coated implantable medical device can further be coated with lubricant to facilitate the transport of implantable medical device (e.g., balloon) through introducer and blood vessels. A lubricant is a substance, usually organic, introduced to reduce friction between surfaces in mutual contact, which ultimately reduces the heat generated when the surfaces move.

In some embodiments, the lubricant is a mineral oil. The term “mineral oil” is used to refer to lubricating base oils derived from crude oil. The American Petroleum Institute (API) designates several types of lubricant base oil, including group I, Group II, Group III, group IV, and Group V mineral oils. Lubricants can also be categorized as paraffinic, naphthenic, aromatic, and synthetic oils. Aqueous lubricants such as polyethylene glycol, bio-lubricants such as vegetable oil, and whale oil are also suitable lubricants.

Petroleum-derived lubricant can also be produced using synthetic hydrocarbons (derived ultimately from petroleum), “synthetic oils”. These include poly α-olefins, synthetic esters, polyalkylene glycols, phosphate esters, alkylated naphthalenes, silicate esters, ionic fluids, and multiply alkylated cyclopentanes.

In some embodiments, a dry lubricant is used. Polytetrafluoroethylene (PTFE) is commonly used as a coating layer on, for example, cooking utensils to provide a non-stick surface. Graphite, hexagonal boron nitride, molybdenum disulfide and tungsten disulfide are examples of solid lubricants.

### Methods of Coating a Medical Device

Another aspect of the present invention provides a method of coating an implantable medical device. This provides a coating procedure that can be used to incorporate nanoparticles in a layer-by-layer fashion. The method includes the steps of a) applying a pharmaceutical composition dispersed in an aqueous solution to the surface of an implantable medical device, wherein the pharmaceutical composition comprises a polymeric coating composition comprising polymeric nanoparticles dispersed within a polymeric matrix, wherein the polymeric nanoparticles include a first therapeutic agent (e.g., sirolimus) and a second therapeutic agent (e.g., paclitaxel), b) removing the aqueous solution by drying to form a layer of the pharmaceutical composition on the surface of the implantable medical device, and repeating steps a) and b) to form a coating comprising a plurality of layers on the surface of the implantable medical device. Additional water-soluble drugs or biological agents can be easily incorporated into the pharmaceutical coating composition during the coating procedure. The polymeric matrix and polymeric nanoparticles can be formed using any of the polymers described herein. For example, in some embodiments, the polymeric matrix comprises polyvinyl alcohol (PVA).

Nanoparticles and drugs that are water soluble or biological agents can be coated either alone or together in layers or between layers. In addition, one of the advantages of the method is that it is useful for drugs which are highly insoluble and can precipitate on an implantable medical device (e.g., a balloon) following coating as large crystalline material, which may limit how much drug can be coated which will not be an issue with the developed coating procedure.

A variety of methods can be used to coat an implantable medical device with a pharmaceutical coating composition. Examples of coating methods include spray coating, dip coating, and jet spraying a dry/dispersion of nanoparticles. An early step in coating the surface of an implantable medical device can be making the surface micro/nanoporous to facilitate embedding the nanoparticles on the surface. However, the use of dip coating preferred, since it is significantly more efficient than over spray coating method, which can result in a significant loss of coating material.

The present invention also contemplates an alternative is coating, which involves preparing a film of PVA loaded with nanoparticles and then wrap it around the implantable medical device. There could be multiple layers of the wraps around the implantable medical device (e.g., a balloon). Film casting is a simple evaporation method.

In some embodiments, the coating method further comprises the step of applying a base coat of polymer matrix without polymeric nanoparticles to the surface of the implantable medical device before conducting step a). The base coat can be made of any of the polymers described herein as being suitable for use in the polymer matrix, including PVA.

In additional embodiments, the coating method further comprising applying an outer coat of polymer matrix without polymeric nanoparticles to the surface of the implantable medical device after forming a coating comprising a plurality of layers on the implantable medical device. The outer coat can be made of any of the polymers described herein as being suitable for use in the polymer matrix, including PVA. Alternately, an outer layer of polymer matrix film can be wrapped around the coated implantable medical device. The outer layer of polymer matrix can help decrease any washout effects. In some embodiments, the outer layer further comprises a sugar and glycerin.

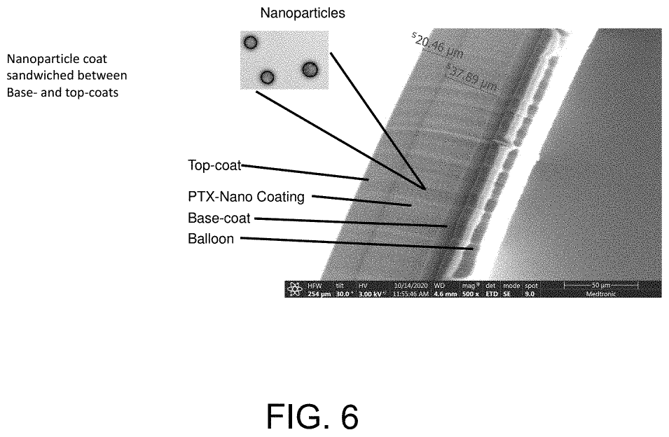

In some embodiments, both base-coat and top-coat are applied, sandwiching the nanoparticle coat (FIG. 6). The number of coats of each layer can be controlled to achieve a particular release profile of the coated nanoparticle layer.

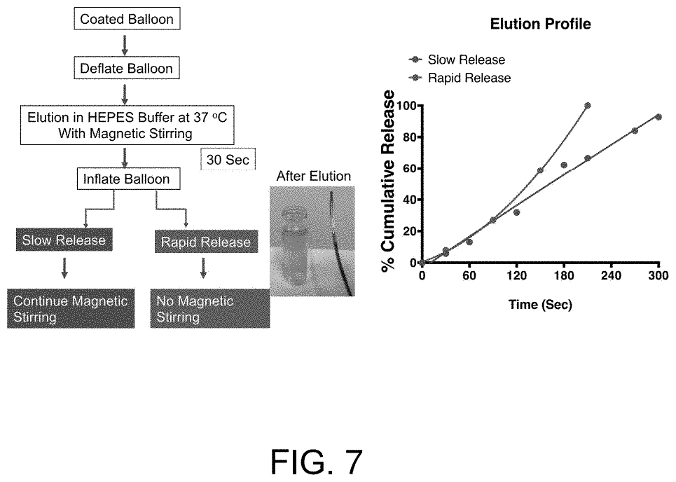

In some embodiment, sugar is added in the coating matrix to achieve slow or faster dissociation of coated nanoparticles (FIG. 7). Reconstitution buffers which are used to reconstitute lyophilized formulation contains different amount of sugar.

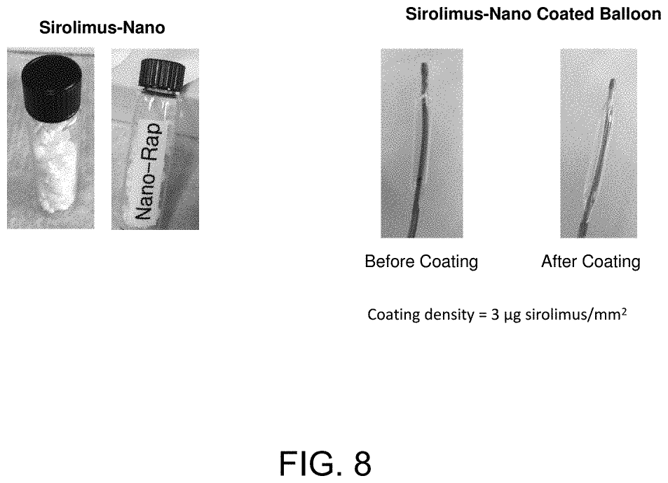

In some embodiment, sirolimus only encapsulated nanoparticles are coated using the coating matrix onto a balloon (FIG. 8).

Medical devices could be of different materials, polymer composition, surface functionality, physical texture, smooth or with pits, holes, groves, striations to accommodate coating matrix containing nanoparticles or drug, or drug+nanoparticles, inflating/deflating devices to facilitate the release of the coating matrix and transport to the target tissue. The formulation of nanoparticles and additives could be integrated into the material of medical devices to achieve certain properties. For example, these could be integrated into stent material if they are made with biodegradable polymers. Composite structure could also be explored to prevent thrombosis as well to achieve antiproliferative effect, promote healing. Inside and outside of stents could be coated with coating copositive to achieve different functionality. For example, outside of stent could be coated with antiproliferative drugs to prevent restenosis whereas inside of stent to prevent thrombosis and to promote reendothelialization. Similarly, balloons could be coated so that outer layer contains antiproliferative drug whereas inner layer contains agents to promote re-endothelialization.

Medical devices can be coated with coating matrix consisting of layers of coating matrix with different composition (e.g., layers containing nanoparticles with different API), inner and outer layers (e.g., stents) can coated with nanoparticles achieving different functionality. For example, the outer layer of stent can contain nanoparticles with antiproliferative agent whereas the inner layer containing nanoparticles to promote re-endothelialization.

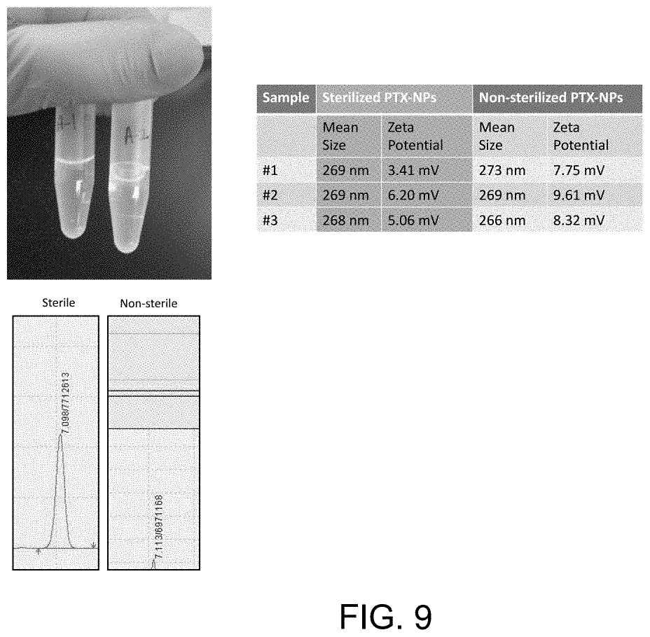

Sterilization is an important step and ensuring that this step does not impact nanoparticle formulation or the encapsulated therapeutic. In this case, paracetic acid-based sterilization method was tested. In this method of sterilization, medical devices are exposed vaporized paracetic acid. The coated balloons and nanoparticles were sterilized using the above technique at RAVOX sterilization solution. The data show in effect of sterilization on nanoparticle characteristics or the encapsulated API (FIG. 9).

### Methods of Treatment

Another aspect of the invention provides a method of treating vascular disease in a subject. The method includes placing a coated implantable medical device into a blood vessel of a subject, such as a human subject, having vascular disease, wherein at least a portion of the implantable medical device is coated with pharmaceutical composition, comprising a polymeric coating composition comprising polymeric nanoparticles dispersed within a polymeric matrix, wherein the polymeric nanoparticles include a first therapeutic agent (e.g., sirolimus) and a second therapeutic agent (e.g., paclitaxel). The polymer matrix and polymeric nanoparticles can include any of the materials described herein for these components of the polymeric coating composition.

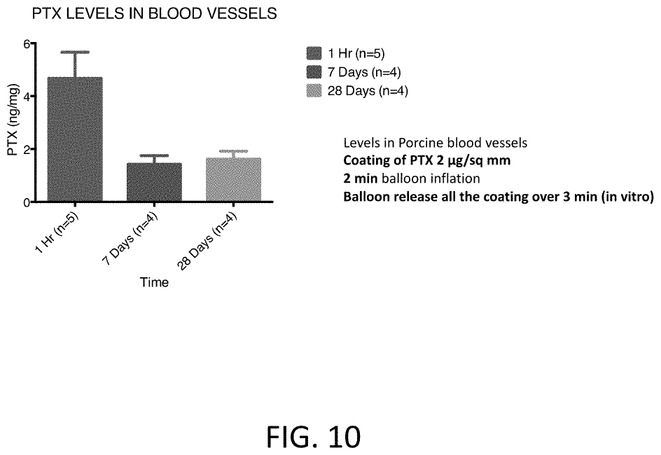

In view of the embodiment described above, the balloons coated with paclitaxel-loaded nanoparticles show sustained retention of paclitaxel in porcine peripheral artery. (FIG. 10).

In addition to delivery to a blood vessel, other routes of administration can be used, particularly intravenous, intra-arterial, transdermal, and intramuscular, delivery into body cavity, etc. The body cavity can be an artery such as coronary, infringingly, aortoiliac, subclavian, mesenteric, basilar and renal. It can also be a urethra, bladder, ureters, esophagus, stomach, colon, trachea, bronchi or alveoli, bile duct.

A common issue in vascular disease is the excessive proliferation of cells. Excessive proliferation of cells and turnover of cellular matrix contribute significantly to the pathogenesis of several diseases, including cancer, atherosclerosis, rheumatoid arthritis, psoriasis, idiopathic pulmonary fibrosis, pancreatic cancer, biliary duct metastases, scleroderma and cirrhosis of the liver. In addition, the excessive proliferation that is triggered by injury.

An example of a vascular disease involving the excessive proliferation of cells is restenosis. Restenosis is the recurrence of stenosis, a narrowing of a blood vessel, leading to restricted blood flow. Restenosis usually pertains to an artery or other large blood vessel that has become narrowed, received treatment to clear the blockage and subsequently become re-narrowed. Accordingly, restenosis is a common adverse event of endovascular procedures. Damage to the blood vessel wall by angioplasty triggers physiological response that can be divided into two stages. The first stage that occurs immediately after tissue trauma, is thrombosis. A blood clot forms at the site of damage and further hinders blood flow, and is accompanied by inflammation. The second stage tends to occur 3-6 months after surgery and is the result of proliferation of cells in the media, a smooth muscle wall in the vessel. Accordingly, in some embodiments, the vascular disease is restenosis.

The invention provides a method of treating vascular disease in a subject. Vascular disease is a class of diseases of blood vessels, including arteries and veins. Vascular disease is typically a pathological state of large and medium muscular arteries and is triggered by endothelial cell dysfunction. Examples of vascular disease include erthromelagia, peripheral artery disease, coronary artery disease, renal artery stenosis, Buerger's disease, Raynaud's disease, disseminated intravascular coagulation, and cerebrovascular disease.

In some embodiments, the vascular disease is peripheral artery disease. Peripheral artery disease occurs when atheromatous plaques build up in the arteries that supply blood to the arms and legs, plaque causes the arteries to narrow or become blocked.

In some embodiments, the vascular disease is coronary artery disease. Coronary artery disease involves the reduction of blood flow to the heart muscle due to build-up of plaque (atherosclerosis) in the arteries of the heart. Examples of coronary artery disease include angina, myocardial infarction, and sudden cardiac death.

### Formulation and Administration

In some embodiments, the nanoparticles including the first and second therapeutic agents are delivered in a pharmaceutical composition coating a medical device such as an implantable medical device, as described herein. For example, the nanoparticles can be provided on coated stents or other devices as listed above (See FIG. 3); such as coated balloons (e.g., infusion balloon; double balloon catheter for localized infusion). However, other forms of delivery of the pharmaceutical compositions include use of a transluminal local drug delivery device or transendocardial delivery system; intravascular or intra-arterial, cerebrovascular delivery using infusion catheters; periadventitial delivery; perivascular delivery; direct injection into arterial wall; intravenous/intra-arterial injection; intravenous/intra-arterial infusion; localized tissue injection near affected blood vessel; microneedle patches; and administration using a microneedle injection balloon.

The following examples are included for purposes of illustration and are not intended to limit the scope of the invention.

### EXAMPLES

### Example 1: Method to Coat Balloon with Functionalized Nanoparticles Containing API

Coating procedure developed allows flexibility of modulating the dose of nanoparticles and hence the drug that can be coated onto the balloon.

Polyvinyl Alcohol (PVA) solution with functionalized nanoparticles dispersed into it is used for coating balloon; the coating procedure is layer-by-layer deposition. This coating method can allow us to achieve uniform or if desired gradient coating of nanoparticles (higher amount in inside layer and lower in outside layer or vice versa) or an outside layer of PVA without nanoparticles to minimize the washout effect prior to deployment of the balloon at the target vessel. PVA is inert and has been used previously in humans for other applications. The coating seems to remain associated with the balloon (based on observation with the coated balloon left at room temperature). There is no observable peeling of the coating from the balloon. See FIGS. 3-5.

In another coating method, lyophilized nanoparticles with PVA in reconstituted in reconstitution buffer that contains sugar and glycerin (Table 1). The reconstituted dispersion has enough PVA that gives require consistency for coating onto the balloon.

### Composition of Base and Topcoat

A 10% w/v PVA solution in water is prepared. Typically, 100 ml solution is prepared but could be less (˜25 ml) or depending upon the need. 10 g PVA is sprinkled slowly into 100 ml Milli-Q sterile water in a beaker while stirring on a magnetic stir plate at 300 rpm at room temperature. After about ˜1 Hr of stirring, water temperature is raised to 85° C. while stirring and with the beaker covered with an aluminum foil. After ˜1 Hr, solution becomes translucent. After cooling to room temperature, PVA solution is filtered through a filtration flask (Fisher Scientific) under vacuum. For each ml of PVA solution as prepared above, glycerin is added and stirred on a magnetic stir plate at ˜300 rpm for 10-15 min.

The base coat is applied to facilitate the subsequent coating of the nanoparticle layer. The basecoat could potentially hold the nanoparticle in the tissue as they are transferred to the tissue due to the bioadhesive nature of PVA (FIG. 6). The above composition as such was used as a basecoat and 1 layer was applied prior to nanoparticle coatings.

The PVA solution described above can be diluted depending upon the desired lag-phase (FIG. 5). More dilute the solution, shorter is the lag phase prior to the release of coated nanoparticle layer from the balloon. The topcoat could also act as a protective layer to the nanoparticle coat (FIG. 6). The above composition as such or 1:1 v/v dilution in water was used as a topcoat. One to 2 layers were applied as a topcoat.

The coating composition including the polymeric nanoparticle is between the topcoat and the basecoat. Layers of coating (basecoat, nanoparticle coat, and topcoat) were determined by cross cutting the balloon and analyzing using scanning electron microscope (FIG. 6). The images show three distinct coating layers. The coated stent surface was characterized by scanning electron microscope. The images show the presence of nanoparticles.

Balloons are taken into a chemical hood for the coating process. The hood set-up is as follows. A horizontal rod is placed inside the hood to tape balloons. The rod is 10 inches inside from the hood door and 8.5 inches above the hood floor. These conditions are set to achieve streamline airflow that allows balloon coating to dry between each layer. The balloon shaft is taped to the horizontal rod so that the balloon is horizontal in position and facing the hood door. At a time, 10-12 balloons can be taped to the rod for coating.

Nanoparticle preparation at a specific concentration is prepared in water. The concentration of nanoparticles is optimized so that coating is retained onto the balloon and is not too liquid so that it drips. This is indirect optimization of viscosity of the coating so that it is retained onto the balloon. The airflow and water evaporation from coating when the balloons are placed inside the hood gain additional viscosity that prevents dripping.

The nanoparticle dispersion is loaded in a one cc tuberculin syringe that is locked using a three-way valve. The nanoparticle dispersion is loaded into the syringe along the inside wall using a 1 ml pipet so that there is no entrapment of air bubbles. If air bubbles are trapped, they can be removed by tapping the syringe. The syringe is loaded up to a mark so that the balloon is dipped completely and there is no overflow. Tuberculin syringe is suitable for a certain diameter balloons. For larger diameter and longer balloons, glass tubes are fabricated accordingly and marked. Larger diameter syringes could also be used.

Base/topcoat solution is also loaded as above into separate syringe. If the base and topcoat composition is different, they are loaded into two separate syringes. Based on the drug dose in nanoparticle formulation and volume in which it is reconstituted, an estimated total volume that is needed onto each balloon to achieve a particular dose is calculated. With volume taken for each coat, one can estimate the number of coats that may be needed to achieve a particular drug dose. The cumulative amount coated onto the balloon provides the estimate of total coated drug dose.

In this example, the coating is a layer-by-layer process. A single base coat is followed by multiple nanoparticle coats and the final topcoat. It was the observation that the base coat helps in coating subsequent first nanoparticle coat.

The balloon is dipped slowly into the coating material loaded into the syringe. Excess of coating is drained into the syringe by touching the tip of the balloon to the inside wall of the syringe. The volume in the syringe before and after each dip is noted. The difference is used for calculating the volume of coating solution coated onto the balloon for each dip.

In summary, the process is a layer-by-layer coating method with a drying cycle between each coat. This appears to avoid formation of air bubbles. Most likely air bubbles are formed as moisture from deep layers is trying to escape but could not because the top layer has dried and is now acting as a barrier. Placing balloons in horizontal position and flipping upside down for the subsequent coat helps in achieving uniform coating.

A number of variations of this method can be used. In one case, the film of the coating material is casted in a petri dish. It is carefully removed and wrapped around the balloon. The balloon is moistened with water to allow sticking of the film onto the balloon. As an example, base coat solution containing Evans blue dye was casted and wrapped around the balloon. One could cast films containing a base coat, then nanoparticle coat and then top coating. Such a film then can be wrapped around the balloon.

In dip coating protocol described above, one can have a gradient number of nanoparticles in different coating layer than the same amount. For example, the initial layers could have high amount whereas top layers a low amount. If there is a washout effect, the top layer that contains lower number of nanoparticles will be lost and the layer that contains high number of nanoparticles is available for transfer at the target site.

Nanoparticles containing PTX and SRL alone can be prepared separately, mixed in a desired proportion, and used for coating. This option is in addition to the dual combination nanoparticles. Further modification could include addition of different API (vasodilator, agent that promotes healing and endothelization, etc.) can be mixed in the combination for coating onto the balloon.

Alternatively, solubilized drug (PTX+SRL) is added either during making nanoparticle formulation or incorporated into the coating layer. While making nanoparticles with solubilized drug, API and solubilizing agent is added into chloroform prior to emulsification as described above. In another instance, API are dissolved in water and mixed with coating formulation that contains nanoparticles.

The method used for coating for balloons has also been for coating stents. In general, the method can be used for coating different medical devices and with different API or combination of API.

Elution of coated nanoparticles from balloon was determined in suitable buffer at 37° C. In this case, nanoparticles in addition to API contained a small amount of near-infrared dye (FIGS. 3 and 4). The eluted samples were collected at different time points. The signal from nanoparticles was quantified to determine cumulative elution of coated nanoparticles.

In addition to lubricant property of glycerin, it acts as an anti-flaking agent when mixed with nanoparticle or PVA coating layer. Other potential anti-flaking agents are: Anti-flaking agents: tricalcium phosphate, powdered cellulose, magnesium stearate, sodium bicarbonate, sodium ferrocyanide, potassium ferrocyanide, ferrocyanide, bone phosphate (i.e., calcium phosphate), sodium silicate, silicon dioxide, calcium silicate, magnesium trisilicate, talcum powder, sodium aluminosilicate, potassium aluminum silicate, calcium aluminosilicate bentonite, aluminum silicate, stearic acid, polydimethylsiloxane.

### Example 2: Determine the Efficacy of the Combination of Sirolimus and Paclitaxel Encapsulated Nanoparticles in Inhibiting Proliferation of Human Vascular Smooth Muscle Cells

Paclitaxel and Sirolimus: These two drugs are the most used in drug-coated balloons (DCBs) and drug eluting stents to prevent vascular restenosis.

Paclitaxel (PTX): It is a microtubule-stabilizing drug that prevents cell-division, and ultimately causes cell death. Therefore, paclitaxel is considered as a Cytotoxic drug. It is commonly used as a cancer chemotherapeutic agent to kill tumor cells. It is also associated with significant side effects due to its cytotoxic effect.

Sirolimus (SRL): Originally developed and used widely as an immunosuppressant, sirolimus, also known as rapamycin, also has antiproliferative effect. Sirolimus forms a complex with intracellular protein, FKBP12, that blocks the activation of the cell-cycle-specific kinase, thus cell-cycle progression. Therefore, sirolimus is considered as cytostatic drug and is also used in oncology.

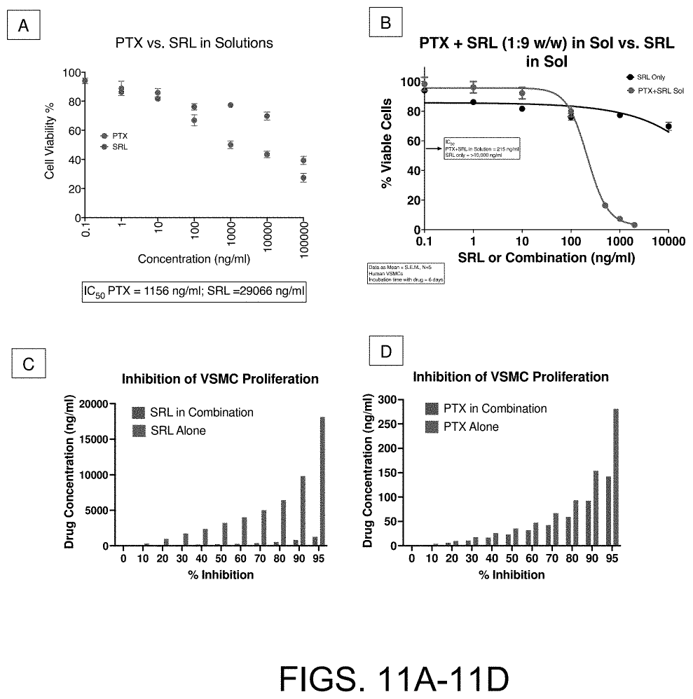

Compared to PTX, therapeutic index of SRL is 1000-fold greater and hence it is a safer drug than PTX. However, the tissue uptake and retention of SRL is significantly lower than PTX. This is because PTX is crystalline whereas SRL is amorphous. Further, PTX taken up by the tissue as crystals dissolves slowly, thus sustaining the drug retention whereas SRL is amorphous, hydrophilic, and unstable. Further PTX binds to the tissue protein hence it is retained in the tissue whereas SRL does not have specific tissue binding sites and hence its retention is transient in the tissue. In this investigation, functionalized nanoparticles encapsulating PTX alone, SRL alone, and combination of both the drugs were formulated. Since, drugs are encapsulated, their tissue uptake and retention are primarily determined by the polymer composition rather than physical characteristics of each drug itself. Another issue with sirolimus is significantly higher dose required than PTX to achieve antiproliferative effect. This invention addresses that issue (FIG. 11A).

Synergistic Effect: Drug synergism happens when the effects of two or more different kinds of drugs are amplified when they are administered jointly. Their impact is greater than their combined effects. The effect is additive when the combined effect is sum of the two drugs and it is antagonistic it is less than the sum effect (FIG. 11B).

Determination of Synergistic Effect of Combination Paclitaxel and Sirolimus: The synergistic effect of the combination treatment was determined in vitro using human vascular smooth muscle cells. The IC₅₀ i.e., the drug amount required to inhibit cell proliferation by 50% with respect to the untreated cells was determined using cell viability assay. Drug combination index (CI) was used to determine if the combination treatment is synergistic (CI=<1), additive (CI=1) or antagonistic (CI>1).

Further invention of the technology lies is determining the combination of SRL and PTX in an optimal ratio in nanoparticle formulation so that the effect is not only synergistic but the dominant mechanism of inhibition of cell proliferation remains cytostatic rather than cytotoxic.

As shown in FIG. 11A, SRL requires significantly higher dose than PTX to achieve the same level of inhibition of cell proliferation. This is because of differences in the mechanisms of action of SRL (cytostatic) and PTX (Cytotoxic). The combination index (CI) 0.15 indicates strong synergism (FIG. 11B).

Objective: To determine the efficacy of the treatment in primary human vascular smooth muscle cells with the drug combination (SRL+PTX, 9:1 w/w) encapsulated within the same nanoparticle formulation as compared to the individual drug (SRL or PTX) encapsulated in separate nanoparticle formulation.

Method: Primary human vascular smooth muscle cells (VSMCs) were incubated with different doses of the encapsulated drug(s) for six days. Cell viability was determined using CyQUANT™ NF Cell Proliferation Assay kit (Cat #C35006, ThermoFisher Scientific). Fluorescent readings were taken by a microplate reader (Cytation 5, BioTek Instruments Inc., Vermont). Percent cell inhibition was calculated with respect to the cells that did not receive any treatment.

Results: Significantly higher doses of drugs (SRL or PTX) are needed when the cells are treated with the individual drug (SRL or PTX) encapsulated nanoparticles compared to the nanoparticles encapsulating both the drugs. See FIGS. 11C and 11D, and Table 1. The dose-effect is more pronounced with SRL than with PTX. Due to differences in their mechanism of action (cytostatic vs. cytotoxic), SRL requires a significantly higher dose than PTX to achieve the same level of inhibition of cell proliferation. The effect of the combination treatment is more evident at higher inhibition. For example, to achieve 90% inhibition of cell proliferation, an 11.8-fold higher dose of SRL alone encapsulated nanoparticles is required compared to the SRL dose in the combination nanoparticle formulation. For PTX, this difference is about 1.7-fold. It is to be noted that inhibition of cell proliferation is not linear with drug dose; it usually follows logarithmic-sigmoidal correlation, i.e., it takes a lot more drug to achieve higher inhibition of cell proliferation (e.g., from 80% to 90%) than lower inhibition of cell proliferation (e.g., 10% to 20%).

TABLE 1
Individual Drugs vs. Combination Drugs

| | Combination | | | Separate | |
|---|---|---|---|---|---|
| % | Formulation (ng/ml) | | | Formulations (ng/ml) | |
| Inhibition | SRL | PTX | Total | SRL Alone | PTX Alone |
| 10 | 17 | 1.9 | 18.9 | 327 | 3.6 |
| 20 | 56 | 6.2 | 62.2 | 991 | 10.2 |
| 30 | 99 | 11.0 | 110 | 1745 | 17.7 |
| 40 | 151 | 16.8 | 167.8 | 2407 | 26.1 |
| 50 | 209 | 23.2 | 232.2 | 3235 | 35.9 |
| 60 | 289 | 32.1 | 321.1 | 4038 | 47.7 |
| 70 | 386 | 42.7 | 428.7 | 5041 | 66.9 |
| 80 | 531 | 59.3 | 590.3 | 6457 | 93.7 |
| 90 | 832 | 92.5 | 924.5 | 9806 | 153.9 |
| 95 | 1283 | 142.6 | 1425.6 | 18120 | 281.2 |

In a separate experiment, VSMCs were treated with the drug in combination in encapsulated in nanoparticles and equivalent doses of each drug formulated in separate nanoparticles. The results show better inhibition of cell proliferation when treated with the nanoparticles encapsulating both the drugs (SRL: PTX, 9:1 w/w) than when treated with PTX-NPs or SRL-NPs with the equivalent dose present in the combination formulation. The combination treatment containing 200 ng/ml (10% PTX and 90% SRL) in nanoparticle formulation shows ˜40% inhibition of cell proliferation whereas 20 ng of PTX-NPs (the amount encapsulated in combination formulation) shows ˜20% inhibition of cell proliferation whereas 180 ng SRL-NPs (the amount encapsulated in combination formulation) shows only ˜5% inhibition of cell proliferation.

Efficacy of encapsulated API: The cells were treated with different doses of sirolimus or paclitaxel, either encapsulated in nanoparticles or as a solution to determine IC₅₀ (concentration of drug that shows 50% inhibition in cell proliferation) for each treatment. The results show that the encapsulated drugs have lower IC₅₀ than the solution (˜3 times lower for sirolimus and ˜1.34 for paclitaxel). Furthermore, considering that nanoparticles are sustained release formulation, and only a fraction of the encapsulated drug is released during the six-day experimental time in cell culture, the enhancement effect is much more pronounced with encapsulation than what data demonstrate.

Conclusions: Encapsulation of drugs enhances their antiproliferative effect. Overall data show a synergistic effect when both the drugs are encapsulated in a single nanoparticle formulation.

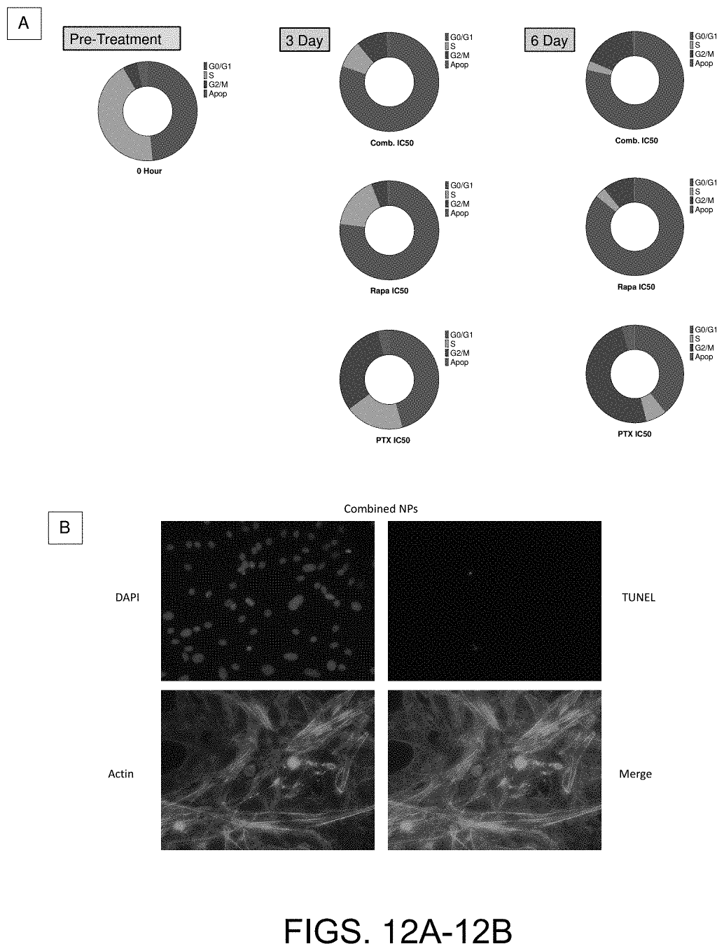

Mechanism of inhibition of cell proliferation with combination treatment: Flow cytometric analysis of the cells treated either with PTX or SRL and Combination was carried out to determine the dominant mechanism of action of the combination treatment on inhibition of cell proliferation. The dominant mechanism of action with combination product is via cell-cycle arrest (FIG. 12). The combination treatment did not cause cell death (cytotoxic effect), maintained cell structure yet inhibited cell proliferation (cytostatic effect) at significantly lower dose than SRL-alone treated cells. The combination dose was ˜14-fold lower than SRL alone dose, suggesting that a small dose of PTX in the combination (PTX:SRL ratio 1:9) treatment boosts the effect of SRL.

The complete disclosure of all patents, patent applications, and publications, and electronically available materials cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A pharmaceutical composition, comprising a polymeric coating composition comprising polymeric nanoparticles dispersed within a polymeric matrix, wherein the polymeric nanoparticles include a first therapeutic agent and a second therapeutic agent.

2. The pharmaceutical composition of claim 1, wherein the polymeric nanoparticles comprise poly(lactic-co-glycolic acid) (PLGA).

3. The pharmaceutical composition of claim 1, wherein the polymeric matrix comprises polyvinyl alcohol (PVA).

4. The pharmaceutical composition of claim 1, wherein the polymeric nanoparticles are surface-functionalized.

5. The pharmaceutical composition of claim 4, wherein the polymeric nanoparticles are surface-functionalized with PVA and poly-1-lysine (PLL).

6. The pharmaceutical composition of claim 5, wherein the polymeric nanoparticles are further surface functionalized with a cationic surfactant.

7. The pharmaceutical composition of claim 5, wherein the polymeric nanoparticle has a cationic zeta potential.

8. The pharmaceutical composition of claim 1, wherein the polymeric nanoparticles have a hydrodynamic diameter from 50 to 1000 nanometers.

9. The pharmaceutical composition of claim 1, wherein the first and second therapeutic agents are both antiproliferative agents.

10. The pharmaceutical composition of claim 9, wherein the first therapeutic agent is a limus compound, and the second therapeutic agent is a taxane compound.

11. The pharmaceutical composition of claim 10, wherein the first therapeutic agent is sirolimus, and the second therapeutic agent is paclitaxel.

12. The pharmaceutical composition of claim 11, wherein the weight ratio of paclitaxel to sirolimus ranges from about 8:1 w/w to about 10:1 w/w.

13. The pharmaceutical composition of claim 11, wherein the weight ratio of sirolimus to paclitaxel ranges from about 8:1 w/w to about 10:1 w/w.

14. The pharmaceutical composition of claim 11, wherein the weight ratio of sirolimus to paclitaxel ranges from about 1:2 w/w to about 2:1 w/w.

15. The pharmaceutical composition of claim 11, wherein the sirolimus and/or the paclitaxel is amorphous.

16. The pharmaceutical composition of claim 1, wherein the polymeric nanoparticles further comprise L-(+)-tartaric acid dimethyl ester or a fatty acid.

17. The pharmaceutical composition of claim 1, wherein the pharmaceutical composition further comprises an imaging agent.

18. The pharmaceutical composition of claim 1, wherein the first and second therapeutic agents are present in different polymeric nanoparticles.

19. The pharmaceutical composition of claim 1, wherein the first and second therapeutic agents are present within the same polymeric nanoparticles.

20. The pharmaceutical composition of claim 1, wherein the first therapeutic agent and/or the second therapeutic agent is solubilized.

21. A coated implantable medical device, wherein at least a portion of the implantable medical device is coated with pharmaceutical composition, comprising a polymeric coating composition comprising polymeric nanoparticles dispersed within a polymeric matrix, wherein the polymeric nanoparticles include a first therapeutic agent and a second therapeutic agent.

22. The coated implantable medical device of claim 21, wherein the implantable medical device is a stent or a balloon catheter.

23. The coated implantable medical device of claim 21, wherein the polymeric nanoparticles comprise poly(lactic-co-glycolic acid) (PLGA).

24. The coated implantable medical device of claim 21, wherein the polymeric matrix comprises polyvinyl alcohol (PVA).

25. The coated implantable medical device of claim 21, wherein the polymeric nanoparticles are surface-functionalized with PVA and poly-1-lysine (PLL).

26. The coated implantable medical device of claim 21, wherein the polymeric nanoparticles have a hydrodynamic diameter from 50 to 1000 nanometers.

27. The coated implantable medical device of claim 21, wherein the pharmaceutical composition is coated with an outer layer of polyvinyl alcohol.

28. The coated implantable medical device of claim 27, wherein the outer layer further comprises a sugar and glycerin.

29. A method of treating vascular disease in a subject, comprising placing a coated implantable medical device into a blood vessel of a subject having vascular disease, wherein at least a portion of the implantable medical device is coated with pharmaceutical composition, comprising a polymeric coating composition comprising polymeric nanoparticles dispersed within a polymeric matrix, wherein the polymeric nanoparticles include a first therapeutic agent and a second therapeutic agent.

30. The method of claim 29, wherein the vascular disease is peripheral artery disease.

31. The method of claim 29, wherein the vascular disease is restenosis.

32. The method of claim 29, wherein the vascular disease is coronary artery disease.

33. The method of claim 29, wherein the implantable medical device is a stent or a balloon catheter.

34. The method of claim 29, wherein the polymeric nanoparticle comprises poly(lactic-co-glycolic acid) (PLGA).

35. The method of claim 29, wherein the polymeric matrix comprises polyvinyl alcohol (PVA).

36. The method of claim 29, wherein the polymeric nanoparticles are surface-functionalized with PVA and poly-1-lysine (PLL).

37. The method of claim 36, wherein the outer layer further comprises a sugar and glycerin.

38. The method of claim 29, wherein the first therapeutic agent is sirolimus and the second therapeutic agent is paclitaxel.

39. The method of claim 29, wherein the pharmaceutical composition is coated with an outer layer of polyvinyl alcohol.

40. The method of claim 29, wherein the subject is human.

41. A method of coating an implantable medical device, comprising
a) applying a pharmaceutical composition dispersed in an aqueous solution to the surface of an implantable medical device, wherein the pharmaceutical composition comprises a polymeric coating composition comprising polymeric nanoparticles dispersed within a polymeric matrix, wherein the polymeric nanoparticles include a first therapeutic agent and a second therapeutic agent,
b) removing the aqueous solution by drying to form a layer of the pharmaceutical composition on the surface of the implantable medical device,
repeating steps a) and b) to form a coating comprising a plurality of layers on the surface of the implantable medical device.

42. The method of claim 41, further comprising the step of applying a base coat of polymer matrix without polymeric nanoparticles to the surface of the implantable medical device before conducting step a).

43. The method of claim 41, wherein the polymeric matrix comprises polyvinyl alcohol (PVA).

44. The method of claim 41, further comprising applying an outer coat of polymer matrix without polymeric nanoparticles to the surface of the implantable medical device after forming a coating comprising a plurality of layers on the implantable medical device.

45. The method of claim 44, wherein the outer layer further comprises a sugar and glycerin.

46. The method of claim 41, wherein the implantable medical device is a stent or a balloon catheter.
